(19) United States
(12) Patent Application Publication
(10) Pub. No.: US 2023/0375539 A1
(43) Pub. Date: Nov. 23, 2023

(54) COMPOSITIONS, SYSTEMS, AND METHODS RELATED TO A DUAL-AFFINITY RATIOMETRIC QUENCHING BIOASSAY

(71) Applicant: North Carolina State University, Raleigh, NC (US)

(72) Inventors: Stefano Menegatti, Raleigh, NC (US); Michael Daniele, Raleigh, NC (US); Katie Kilgour, Raleigh, NC (US); Brendan Turner, Raleigh, NC (US)

(21) Appl. No.: 18/248,296

(22) PCT Filed: Oct. 8, 2021
(86) PCT No.: PCT/US2021/054245

§ 371 Date: Apr. 7, 2023

Related U.S. Application Data
(60) Provisional application No. 63/089,806, filed on Oct. 9, 2020.

(51) Int. Cl. G01N 33/542; G01N 33/68

(52) U.S. Cl. CPC G01N 33/542; G01N 33/6854; G01N 2021/6439

(57) ABSTRACT

The present disclosure provides compositions, methods, and systems related to a dual-affinity ratiometric quenching bioassay. In particular, the present disclosure provides novel compositions and methods that combine selective biorecognition and quenching of fluorescence signals for rapid and sensitive quantification of antibodies in complex samples.

### CROSS REFERENCE TO RELATED APPLICATIONS

This application claims priority to and the benefit of U.S. Provisional Patent Application No. 63/089,806 filed Oct. 9, 2020, which is incorporated herein by reference in its entirety for all purposes.

### FIELD

The present disclosure provides compositions, methods, and systems related to a dual-affinity ratiometric quenching bioassay. In particular, the present disclosure provides novel compositions and methods that combine selective biorecognition and quenching of fluorescence signals for rapid and sensitive quantification of antibodies in complex samples.

### BACKGROUND

Accurate quantification of monoclonal antibodies (mAbs) in complex media, such as the serum of patients undergoing immunotherapy, industrial cell culture harvests and streams, and fluids derived from transgenic plants and animals is paramount to ensure the health of patients and product quality in biopharmaceutical industry. To date, a myriad of assays have been developed for antibody detection and measurement, including lateral flow assays, ELISA, and immunoaffinity chromatography. These, however, differ widely in terms of duration (from minutes to hours), performance (sensitivity, limit of detection, and dynamic range), and cost. Likewise, the biorecognition moieties utilized to target the antibody analyte include protein-based affinity tags such as antigens, secondary antibodies and antibody-binding receptors (e.g., Protein A, Protein G, and Fc receptors FcγRs), as well as synthetic affinity tags such as aptamers and peptides. Finally, detection modalities vary significantly ranging from optical (e.g., UV/vis, fluorescence, and surface plasmon resonance) to electrochemical (e.g., impedance and amperometry) and acoustic (e.g., photoacoustic and quartz crystal microbalance). Fluorescence holds a preeminent place among detection modalities, owing to its high sensitivity, flexibility, and availability of fluorescence spectrophotometers. The generation of a fluorescence signal by the affinity tags can be accomplished either by chemical conjugation (e.g., by labeling them with synthetic fluorophores), or enzymatically by fusing them with enzymes (e.g., horseradish peroxidase or luciferase) that convert substrates into fluorescent products. Of major interest are combinations of fluorophores and labelling strategies that can engage in phenomena such as static or dynamic quenching and energy transfer. Such combinations of fluorophores and labelling strategies enable continuous monitoring of the titer of analytes as their concentration evolves with time and provide information regarding the biomolecular structure of the analyte-tag complex.

### SUMMARY

Embodiments of the present disclosure include a ratiometric bioassay method for detecting a target antibody in a sample. In accordance with these embodiments, the method includes combining an antigen coupled to a first fluorophore, an antibody-binding moiety coupled to a second fluorophore, and a sample comprising or suspected of comprising a target antibody, exposing the antigen coupled to the first fluorophore, the antibody-binding moiety coupled to the second fluorophore, and the sample to fluorescent light comprising an excitation wavelength of the first and/or the second fluorophore, and detecting fluorescence emission from the first and/or the second fluorophore. In some embodiments, the fluorescence emission from the first and the second fluorophore is reduced, and wherein the reduced fluorescence emission is proportional to the antibody concentration in the sample.

In some embodiments, the antigen is capable of being bound by the antibody in the sample.

In some embodiments, the antibody-binding moiety comprises a polypeptide capable of binding a region of the target antibody that does not comprise the antigen-binding site. In some embodiments, the antibody binding moiety comprises Protein L, Protein A, or Protein G. In some embodiments, the antibody binding moiety is Protein L.

In some embodiments, the first fluorophore is a high-energy fluorophore and the second fluorophore is a low-energy fluorophore. In some embodiments, the second fluorophore is a high-energy fluorophore and the first fluorophore is a low-energy fluorophore.

In some embodiments, the high-energy fluorophore is selected from the group consisting of Fluorescein, Oregon Green 488, Oregon Green 514, Rhodamine Green, Rhodamine Green-X, Eosin, 4′, 6-diamidino-2-phenylindole, Alexafluor 405, Alexafluor 350, Alexafluor 500, Alexafluor 488, Alexafluor 430, Alexafluor 514, and Alexafluor 532.

In some embodiments, the low-energy fluorophore is selected from the group consisting of Rhodamine, Rhodamine B, Rhodamine Red-X, Tetramethylrhodamine, Lissamine, Texas Red and Texas Red-X, Naphthofluorescein, Carboxyrhodamine 6G, Alexafluor 555, Alexafluor 546, Alexafluor 568, Alexafluor 594, Alexafluor 610, Alexafluor 633, Alexafluor 635, Alexafluor 647, Alexafluor 660, Alexafluor 680, Alexafluor 700, and Alexafluor 750.

In some embodiments, the antigen is coupled to fluorescein or Alexafluor 350. In some embodiments, the antibody-binding moiety is coupled to Rhodamine.

In some embodiments, the method further includes determining the concentration of the target antibody in the sample based on the reduction in fluorescence emission of the first and/or the second fluorophore.

In some embodiments, the method further includes incubating the sample comprising or suspected of comprising the target antibody, the antigen coupled to the first fluorophore, and the antibody-binding moiety coupled to the second fluorophore for 30 minutes or less prior to measuring the fluorescent emission of the first and/or the second fluorophore.

In some embodiments, the method further includes incubating the sample comprising or suspected of comprising the target antibody, the antigen coupled to the first fluorophore, and the antibody-binding moiety coupled to the second fluorophore for 10 minutes or less prior to measuring the fluorescent emission of the first and/or the second fluorophore.

In some embodiments, the antigen is coupled to the first fluorophore at an antigen-to-fluorophore ratio of about 1:0.2 to about 1:20.

In some embodiments, the antibody-binding moiety is coupled to the second fluorophore at an moiety-to-fluorophore ratio of about 1:0.2 to about 1:20.

In some embodiments, the first fluorophore and the second fluorophore are present in the composition at a first-to-second fluorophore ratio of about 1:1 to about 1:20.

In some embodiments, the sample is undiluted. In some embodiments, the sample is a whole blood sample, a plasma sample, a serum sample, a urine sample, a saliva sample, a tissue sample, a cell culture sample, a cell lysate sample, or a cell culture media sample.

In some embodiments, the antigen comprises a protein forming the capsid of a virus, or a fragment thereof.

In some embodiments, the antigen comprises the spike (S) protein of the SARS-CoV-2 virus, or a fragment thereof.

Embodiments of the present disclosure also include a composition for performing a ratiometric bioassay to detect an antibody in a sample. In accordance with these embodiments, the composition includes an antigen coupled to a first fluorophore, an antibody-binding moiety coupled to a second fluorophore, and a sample comprising or suspected of comprising a target antibody. In some embodiments, fluorescence emission from the first and the second fluorophore is reduced upon exposure to fluorescent light comprising an excitation wavelength of the first and/or the second fluorophore. In some embodiments, the reduced fluorescence emission is proportional to the antibody concentration in the sample.

In some embodiments, the antigen is capable of being bound by the antibody in the sample.

In some embodiments, the antibody-binding moiety comprises a polypeptide capable of binding a region of the target antibody that does not comprise the antigen-binding site. In some embodiments, the antibody binding moiety comprises Protein L, Protein A, or Protein G.

In some embodiments, the first fluorophore is a high-energy fluorophore and the second fluorophore is a low-energy fluorophore. In some embodiments, the second fluorophore is a high-energy fluorophore and the first fluorophore is a low-energy fluorophore.

In some embodiments, the high-energy fluorophore is selected from the group consisting of Fluorescein, Oregon Green 488, Oregon Green 514, Rhodamine Green, Rhodamine Green-X, Eosin, 4′, 6-diamidino-2-phenylindole, Alexafluor 405, Alexafluor 350, Alexafluor 500, Alexafluor 488, Alexafluor 430, Alexafluor 514, and Alexafluor 532.

In some embodiments, the low-energy fluorophore is selected from the group consisting of Rhodamine, Rhodamine B, Rhodamine Red-X, Tetramethylrhodamine, Lissamine, Texas Red and Texas Red-X, Naphthofluorescein, Carboxyrhodamine 6G, Alexafluor 555, Alexafluor 546, Alexafluor 568, Alexafluor 594, Alexafluor 610, Alexafluor 633, Alexafluor 635, Alexafluor 647, Alexafluor 660, Alexafluor 680, Alexafluor 700, and Alexafluor 750.

In some embodiments, the antigen is coupled to the first fluorophore at an antigen-to-fluorophore ratio of about 1:0.5 to about 1:20.

In some embodiments, the antibody binding moiety is coupled to the second fluorophore at an moiety-to-fluorophore ratio of about 1:0.2 to about 1:20.

In some embodiments, the first fluorophore and the second fluorophore are present in the composition at a first-to-second fluorophore ratio of about 1:1 to about 1:20.

In some embodiments, the sample is undiluted. In some embodiments, sample is a whole blood sample, a plasma sample, a serum sample, a urine sample, a saliva sample, a tissue sample, a cell culture sample, a cell lysate sample, or a cell culture media sample.

In some embodiments, the antigen comprises a protein forming the capsid of a virus, or a fragment thereof.

In some embodiments, the antigen comprises the spike (S) protein of the SARS-CoV-2 virus, or a fragment thereof.

Embodiments of the present disclosure also include a kit for performing a ratiometric bioassay to detect a target antibody in a sample. In accordance with these embodiments, the kit includes an antigen coupled to a first fluorophore, an antibody binding moiety coupled to a second fluorophore, and at least one container.

In some embodiments, the kit further comprises a buffer and/or instructions for performing the bioassay.

In some embodiments, the kit further comprises the target antibody or a fragment or derivative thereof.

### BRIEF DESCRIPTION OF THE DRAWINGS

FIGS. 1A-1D: Crystal structures of the complexes (A) Trastuzumab:^{Rh}PrL (derived from PDB IDs: 4HKZ and 1IGY), (B) Trastuzumab:^{Fl}Her2 (derived from PDB IDs: 60GE and 1IGY), (C) Trastuzumab:2^{Rh}PrL:2^{Fl}Her2 (overlap of panels A and B), and (D) Adalimumab:^{Rh}PrL:^{Fl}TNF-α (derived from PDB IDs: 3WD5, 4HKZ, 1HEZ, and 1IGY). The heavy chain of the mAbs are in light blue, the light chain of the mAbs are in salmon, ^{Rh}PrL is in red, and the antigens ^{Fl}Her2 and ^{Fl}TNF-α are in green. The structures of the complexes were generated by overlapping the crystal structures published on the Protein Data Bank using the open source molecular visualization software PyMOL.

FIG. 2: DARQ assay: a target mAb in solution is incubated with rhodamine-labeled Protein L (^{Rh}PrL) and fluorescein-labeled antigen (^{Fl}AG). Upon formation of the mAb:^{Rh}PrL:^{Fl}AG complex, the fluorescent emission of ^{Rh}PrL and ^{Fl}AG is decreased in a mAb concentration-dependent fashion.

FIGS. 3A-3B: Values of normalized fluorescence (relative fluorescence units, RFU) vs. antibody concentration measured by fluorescence spectrophotometry (λₑₓ=480 nm; λₑₘ=525 nm) of the dual fluorescence affinity complex Trastuzumab:2^{Rh}PrL:2^{Fl}Her2 formed by mixing ^{Rh}PrL and ^{Fl}Her2 at constant stoichiometric amounts with solutions of varying concentrations of Trastuzumab in either (A) PBS (non-competitive conditions) or (B) CHO cell culture harvest (competitive conditions). The red line marks the region of linearity of the fluorescence signal vs. mAb concentration. The vertical blue line marks the point of stoichiometric equivalence, where the concentration of each labeled protein is twice the concentration of antibody.

FIGS. 4A-4B: Values of normalized fluorescence (relative fluorescence units, RFU) vs. antibody concentration measured by fluorescence spectrophotometric analysis (λₑₓ=480 nm; λₑₘ=525 nm) of the dual fluorescence affinity complex Adalimumab:2^{Rh}PrL:2^{Fl}TNF-α formed by mixing ^{Rh}PrL and ^{Fl}TNF-α at constant stoichiometric amounts with solutions of varying concentrations of Adalimumab in either (A) PBS (non-competitive conditions) or (B) CHO cell culture harvest (competitive conditions). The red line marks the region of linearity of the fluorescence signal vs. mAb concentration. The vertical blue line marks the point of stoichiometric equivalence, where the concentration of each labeled protein is twice the concentration of antibody.

FIG. 5: Values of normalized fluorescence (relative fluorescence units, RFU) vs. antibody concentration measured by fluorescence spectrophotometry (λₑₓ=335 nm; λₑₘ=445 nm) of the dual fluorescence affinity complex Adalimumab:2^{Rh}PrL:2^{AF350}TNF-α formed by mixing ^{Rh}PrL and ^{AF350}TNF-α at constant stoichiometric amounts with solutions of varying concentrations of Adalimumab in PBS. The red line marks the region of linearity of the fluorescence signal vs. mAb concentration. The vertical blue line marks the point of stoichiometric equivalence, where the concentration of each labeled protein is twice the concentration of antibody.

FIGS. 6A-6B: Values of normalized fluorescence (relative fluorescence units, RFU) vs. antibody concentration measured by fluorescence spectrophotometry (λₑₓ=480 nm; λₑₘ=525 nm) of the dual fluorescence affinity complex Adalimumab:2^{Rh}PrL:2^{Fl}TNF-α formed by mixing ^{Rh}PrL and ^{Fl}TNF-α at constant stoichiometric amounts with solutions of varying concentrations of Adalimumab at different incubation time (1, 5, 10, and 20 min) in either (A) PBS (non-competitive conditions) or (B) CHO cell culture harvest (competitive conditions). The vertical blue line marks the point of stoichiometric equivalence, where the concentration of each labeled protein is twice the concentration of antibody.

FIGS. 7A-7B: Values of normalized fluorescence (relative fluorescence units, RFU) vs. antibody concentration measured by fluorescence spectrophotometry (λₑₓ=480 nm; λₑₘ=573 nm) of the dual fluorescence affinity complex Trastuzumab:2^{Rh}PrL:2^{Fl}Her2 formed by mixing ^{Rh}PrL and ^{Fl}Her2 at constant stoichiometric amounts with solutions of varying concentration of Trastuzumab in either (A) PBS (non-competitive conditions) or (B) CHO cell culture harvest (competitive conditions). The vertical blue line marks the point of stoichiometric equivalence, where the concentration of each labeled protein is twice the concentration of antibody.

FIGS. 8A-8B: Values of normalized fluorescence (relative fluorescence units, RFU) vs. antibody concentration measured by fluorescence spectrophotometry (λₑₓ=480 nm; λₑₘ=573 nm) of the dual fluorescence affinity complex Adalimumab:2^{Rh}PrL:2^{F}12TNF-α formed by mixing ^{Rh}PrL and ^{Fl}Her2 at constant stoichiometric amounts with solutions of varying concentrations of Trastuzumab in either (A) PBS (non-competitive conditions) or (B) CHO cell culture harvest (competitive conditions). The vertical blue line marks the point of stoichiometric equivalence, where the concentration of each labeled protein is twice the concentration of antibody.

FIGS. 9A-9B: Values of normalized fluorescence (relative fluorescence units, RFU) vs. antibody concentration measured by fluorescence spectrophotomety (λₑₓ=480 nm; λₑₘ=525 nm) of the affinity complexes (A) Trastuzumab:2^{Fl}Her2 and Adalimumab:^{F}12TNF-α and (B) Trastuzumab:2^{Rh}PrL and Adalimumab:2^{Rh}PrL formed in PBS by mixing either ^{Rh}PrL or fluorescein-labeled antigen (^{Fl}Her2 or ^{Fl}TNF-α) with the corresponding mAb. The vertical blue line marks the point of stoichiometric equivalence, where the concentration of each labeled protein is twice the concentration of antibody.

FIGS. 10A-10B: Values of normalized fluorescence (relative fluorescence units, RFU) vs. antibody concentration measured by fluorescence spectrophotometry of the affinity complex Adalimumab:2PrL:2_{AF350}TNF-α at (A) λₑₓ=335 nm; λₑₘ=445 nm, and (B) λₑₓ=335 nm; λₑₘ=573 nm. The vertical blue line marks the point of stoichiometric equivalence, where the concentration of each labeled protein is twice the concentration of antibody.

FIGS. 11A-11C: Values of normalized fluorescence (relative fluorescence units, RFU) vs. antibody concentration measured by fluorescence spectrophotometry of the affinity complex Adalimumab:2^{Rh}PrL:2TNF-α at the excitation and emission wavelengths of (A) λₑₓ=480 nm; λₑₘ=573 nm, (B) λₑₓ=480 nm; λₑₘ=525 nm, and (C) λₑₓ=525 nm; λₑₘ=573 nm. The vertical blue line marks the point of stoichiometric equivalence, where the concentration of each labeled protein is twice the concentration of antibody.

FIGS. 12A-12B: Values of normalized fluorescence (relative fluorescence units, RFU) vs. antibody concentration measured by fluorescence spectrophotometry of the affinity complex Adalimumab:2^{Rh}PrL:2^{AF350}TNF-α at the excitation and emission wavelengths of (A) λₑₓ=335 nm; λₑₘ=573 nm, (B) λₑₓ=525 nm; λₑₘ=573 nm. The vertical blue line marks the point of stoichiometric equivalence, where the concentration of each labeled protein is twice the concentration of antibody.

FIGS. 13A-13C: Values of normalized fluorescence (relative fluorescence units, RFU) vs. antibody concentration measured by fluorescence spectrophotometric analysis of the affinity complex Adalimumab:2^{Rh}PrL:2^{Rh}TNF-α at the excitation and emission wavelengths of (A) 480 nm and 573 nm, (B) 480 nm and 525 nm, and (C) 525 nm and 573 nm. The vertical blue line marks the point of stoichiometric equivalence, where the concentration of each labeled protein is twice the concentration of antibody.

### DETAILED DESCRIPTION

More than 100 monoclonal antibodies (mAbs) are in industrial and clinical development to treat myriad diseases. Accurate quantification of mAbs in complex media, derived from industrial and patient samples, is vital to determine production efficiency or pharmacokinetic properties. To date, mAb quantification requires time and labor intensive assays. As described further herein, embodiments of the present disclosure include a novel bioassay termed Dual-Affinity Ratiometric Quenching (“DARQ”), which combines selective biorecognition and quenching of fluorescence signals for rapid and sensitive quantification of therapeutic monoclonal antibodies (mAbs). The reported assay relies on the affinity complexation of the target mAb by the corresponding antigens and Protein L (PrL, which targets the Fab region of the antibody), respectively labeled with fluorescein and rhodamine. Within the affinity complex, the mAb acts as a scaffold framing the labeled affinity tags (PrL and antigen) in a molecular proximity that results in ratiometric quenching of their fluorescence emission. Notably, the decrease in fluorescence emission intensity is linearly dependent upon mAb concentration in solution. Control experiments conducted with one affinity tag only, two tags labeled with equal fluorophores, or two tags labeled with fluorophores of discrete absorbance and emission bands exhibited significantly reduced effect. The assay was evaluated in non-competitive (pure mAb) and competitive conditions (mAb in a Chinese Hamster Ovary (CHO) cell culture harvest). The “DARQ” assay is highly reproducible (coefficient of variation ˜0.8-0.7%) and rapid (5 min), and its sensitivity (˜0.2-0.5 ng·mL⁻¹), limit of detection (75-119 ng·mL⁻¹), and dynamic range (300-1600 ng·mL⁻¹) are independent of the presence of CHO host cell proteins.

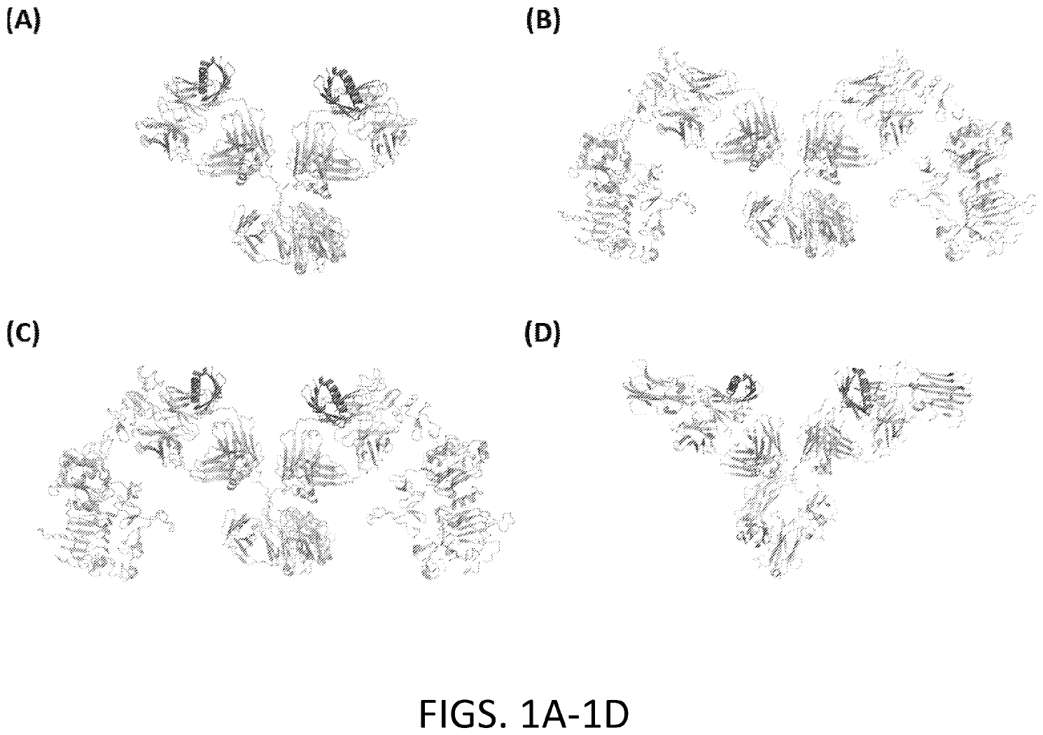

In accordance with these embodiments, the bioassays of the present disclosure involve the formation of a 5-protein affinity complex comprising the target mAb, its two corresponding antigens, and two Protein L (PrL) molecules. PrL is a surface protein derived from Peptostreptococcus magnus and binds the light chain of the Fab region of antibodies, specifically those belonging to the κI, κIII, and κIV subtypes. As described further herein, Trastuzumab (brand name: Herceptin®) and Adalimumab (brand name: Humira®) were used as model κI-mAbs, owing to their demonstrated therapeutic value in fighting cancer and autoimmune diseases. Accordingly, Tumor Necrosis Factor-α (TNF-α) and human epidermal growth factor receptor 2 (Her2) were employed as model antigen (AG) molecules. The fluorophores used herein, namely fluorescein (λₑₓ/λₑₘ: 494/512 nm) and rhodamine (λₑₓ/λₘ: 552/575 nm), were chosen for their expected absorbance-emission spectral overlap and extensive use in fluorescent labeling applications. Specifically, the antigens were labeled with fluorescein (^{Fl}Her2 or ^{Fl}TNF-α); whereas PrL was labeled with rhodamine (^{Rh}PrL). Within the affinity complex, the mAb acts as a molecular scaffold that maintains 2 ^{Rh}PrL and 2 fluorescein-labeled antigen molecules (^{Fl}Her2 or ^{Fl}TNF-α) in a predefined orientation and distance (˜3.10 nm between the centers of mass of PrL and TNF-α, and ˜4.98 nm between the centers of mass of PrL and Her2), as illustrated in FIG. 1.

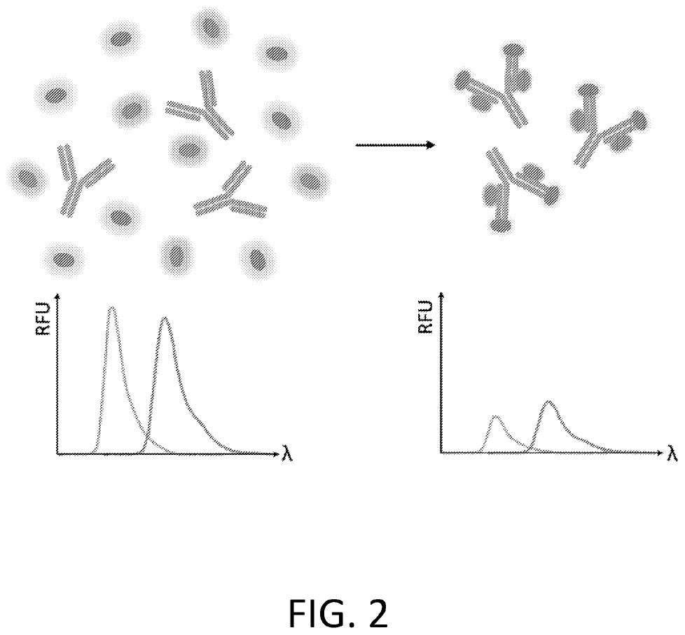

Embodiments of the present disclosure demonstrate that fluorescence emission of both fluorophores decreases linearly with the antibody concentration (FIG. 2). In some cases, the decrease of the fluorescein signal (λₑₘ=525 nm) was more pronounced than that of the rhodamine signal. Without being bound by a particular mechanism, one possible explanation is that the fluorophores displayed on ^{Rh}PrL and ^{Fl}AG in complex with the mAb engage in a quenching mechanism. A set of control experiments was performed comprising different fluorophores and affinity complex schemes. To this end, Alexafluor 350 (AF350, λₑₓ/λₑₘ: 335/445 nm) was adopted as a control fluorophore since it does not exhibit significant spectral overlap with rhodamine, which limits the potential for non-radiative energy transfer. In one embodiment, the control assays included (i) single-affinity labeling by complexing the target antibody with either ^{Rh}PrL or ^{Fl}AG alone; (ii) homogeneous dual-affinity labeling by complexing the Adalimumab using ^{Rh}PrL and ^{Rh}TNF-α; (iii) dual fluorescence—dual affinity labeling by complexing the Adalimumab using ^{Rh}PrL and ^{AF350}TNF-α; and (iv) single fluorescence—dual affinity labeling by complexing the Adalimumab using unlabeled PrL and ^{AF350}TNF-α.

Collectively, the results provided herein demonstrate that the mechanism underlying the bioassays of the present disclosure involve fluorescence quenching, reliant on the labelling of the antigen and PrL as an acceptor-donor pair. The decrease in fluorescein emission with mAb concentration is not due to a traditional FRET mechanism, since the rhodamine signal does not concurrently increase. In some cases, quenching arises predominantly in the high-energy fluorophores (e.g., AF350 and Fl), as observed in the initial assays and control test (iii). However, the exhibited quenching requires the presence of Rh, since control test (i) conducted with ^{Fl}AG alone and (iv) conducted PrL and ^{AF350}TNF-α did not show any decrease in fluorescence signal of either high-energy fluorophore. In some cases, no quenching of the low-energy dye (i.e., Rh) was observed (e.g., in control test (i) with ^{Rh}PrL alone and (iii) with ^{Rh}PrL and ^{Rh}TNF-α).

Additionally, the high affinity and selectivity of the interaction of mAb with PrL and AG molecules provides high sensitivity (˜0.2-0.5 ng·mL⁻¹), low limit of detection (75-119 ng·mL⁻¹), and broad dynamic range (300-1600 ng·mL⁻¹), and eliminates any variability associated to the presence of other proteins in solution. When evaluated in buffered aqueous solution (non-competitive) or in a Chinese Hamster Ovary (CHO) cell culture harvest (competitive), the assay did not exhibit any appreciable variation of performance. Furthermore, the fast kinetics of formation of the mAb:2^{Rh}PrL:2^{Fl}AG complex makes DARQ a rapid (<5 min) and reproducible (coefficient of variation ˜0.8-0.7%) mix-and-read assay with considerably higher throughput compared to commercial mAb-specific ELISA kits (see, e.g., Table 5).

The current technologies utilized by analytical labs at both clinical and biomanufacturing sites for antibody quantification in complex media are laborious and involve expensive consumables or equipment. To overcome these issues, a novel “DARQ” (dual affinity ratiometric quenching) assay was developed for antibody quantification that is rapid, robust, and reproducible, and accurate. The assay relies on a highly selective affinity interaction between the target antibody, the fluorescein-labeled corresponding antigen, and rhodamine-labeled Protein L. The antibody captures the fluorescently labeled proteins, framing them in a supramolecular affinity complex, wherein the fluorophores are constrained in proximity within a dense proteinaceous structure. This translates into a decrease of their fluorescent emission that is linearly dependent upon the antibody concentration. Control assays performed using different combinations of fluorescent affinity tags, while not revealing the specific nature of the mechanism at hand, indicate that it is of the nature of energy transfer quenching. From a technology standpoint, the high binding strength and selectivity of both antigen and Protein L to the target antibody accelerates the formation of the affinity complex, leaving no residual free antibody in solution. The combination of biorecognition and fluorescence quenching makes the assay rapid (˜5 min), highly sensitive (<0.5 ng·mL⁻¹), and reproducible (CoV <1.7%). Reliance on Protein L, which targets the antibody's Fab region of κI, κIII, and κIV subtypes, does not limit the applicability of the assay, given that most therapeutic antibodies currently on the market belong to the κI group. This makes DARQ an ideal mix-and-read assay for at-line monitoring of antibody concentration in bioprocessing fluids (e.g., clarified harvest and chromatographic fractions) or a point-of-care test (POCT) for clinical laboratories.

Section headings as used in this section and the entire disclosure herein are merely for organizational purposes and are not intended to be limiting.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Correlated to” as used herein refers to compared to.

“Coefficient of variation” (CV), also known as “relative variability,” is equal to the standard deviation of a distribution divided by its mean.

“Component,” “components,” or “at least one component,” refer generally to an antigen, an antibody-binding moiety, a target antibody, a fluorophore, a calibrator, a control, a sensitivity panel, a container, a buffer, a diluent, a salt, an enzyme, a co-factor for an enzyme, a detection reagent, a pretreatment reagent/solution, a substrate (e.g., as a solution), a stop solution, and the like that can be included in a kit for assay of a test sample in accordance with the methods described herein and other methods known in the art based on the present disclosure.

“Controls” as used herein generally refers to a reagent whose purpose is to evaluate the performance of a measurement system in order to assure that it continues to produce results within permissible boundaries (e.g., boundaries ranging from measures appropriate for a research use assay on one end to analytic boundaries established by quality specifications for a commercial assay on the other end). To accomplish this, a control should be indicative of patient results and optionally should somehow assess the impact of error on the measurement (e.g., error due to reagent stability, calibrator variability, instrument variability, and the like).

“Quality control reagents” in the context of the assays and kits described herein, include, but are not limited to, calibrators, controls, and sensitivity panels. A “calibrator” or “standard” typically is used (e.g., one or more, such as a plurality) in order to establish calibration (standard) curves for interpolation of the concentration of an analyte, such as an antibody or an analyte. Alternatively, a single calibrator, which is near a reference level or control level (e.g., “low”, “medium”, or “high” levels), can be used. Multiple calibrators (i.e., more than one calibrator or a varying amount of calibrator(s)) can be used in conjunction to comprise a “sensitivity panel.”

“Dynamic range” as used herein refers to range over which an assay readout is proportional to the amount of target molecule or analyte in the sample being analyzed. The dynamic range can be the range of linearity of the standard curve.

“Limit of Blank (LoB)” as used herein refers to the highest apparent analyte concentration expected to be found when replicates of a blank sample containing no analyte are tested.

“Limit of Detection (LoD)” as used herein refers to the lowest concentration of the measurand (i.e., a quantity intended to be measured) that can be detected at a specified level of confidence. The level of confidence is typically 95%, with a 5% likelihood of a false negative measurement. LoD is the lowest analyte concentration likely to be reliably distinguished from the LoB and at which detection is feasible. LoD can be determined by utilizing both the measured LoB and test replicates of a sample known to contain a low concentration of analyte. The LoD term used herein is based on the definition from Clinical and Laboratory Standards Institute (CLSI) protocol EP17-A2 (“Protocols for Determination of Limits of Detection and Limits of Quantitation; Approved Guideline—Second Edition,” EP17A2E, by James F. Pierson-Perry et al., Clinical and Laboratory Standards Institute, Jun. 1, 2012).

“Limit of Quantitation (LoQ)” as used herein refers to the lowest concentration at which the analyte can not only be reliably detected but at which some predefined goals for bias and imprecision are met. The LoQ may be equivalent to the LoD or it could be at a much higher concentration.

“Linearity” refers to how well the method or assay's actual performance across a specified operating range approximates a straight line. Linearity can be measured in terms of a deviation, or non-linearity, from an ideal straight line. “Deviations from linearity” can be expressed in terms of percent of full scale. In some of the methods disclosed herein, less than 10% deviation from linearity (DL) is achieved over the dynamic range of the assay. “Linear” means that there is less than or equal to about 20%, about 19%, about 18%, about 17%, about 16%, about 15%, about 14%, about 13%, about 12%, about 11%, about 10%, about 9%, or about 8% variation for or over an exemplary range or value recited.

“Reference level” as used herein refers to an assay cutoff value that is used to assess diagnostic, prognostic, or therapeutic efficacy and that has been linked or is associated herein with various clinical parameters (e.g., presence of disease, stage of disease, severity of disease, progression, non-progression, or improvement of disease, etc.). It is well-known that reference levels may vary depending on the nature of the assay (e.g., antibodies employed, reaction conditions, sample purity, etc.) and that assays can be compared and standardized. Whereas the precise value of the reference level may vary between assays, the embodiments as described herein should be generally applicable and capable of being extrapolated to other assays.

“Antibody” and “antibodies” as used herein refers to monoclonal antibodies, monospecific antibodies (e.g., which can either be monoclonal, or may also be produced by other means than producing them from a common germ cell), multispecific antibodies, human antibodies, humanized antibodies (fully or partially humanized), animal antibodies such as, but not limited to, a bird (for example, a duck or a goose), a shark, a whale, and a mammal, including a non-primate (for example, a cow, a pig, a camel, a llama, a horse, a goat, a rabbit, a sheep, a hamster, a guinea pig, a cat, a dog, a rat, a mouse, etc.) or a non-human primate (for example, a monkey, a chimpanzee, etc.), recombinant antibodies, chimeric antibodies, single-chain Fvs (“scFv”), single chain antibodies, single domain antibodies, Fab fragments, F(ab′) fragments, F(ab′)₂ fragments, disulfide-linked Fvs (“sdFv”), and anti-idiotypic (“anti-Id”) antibodies, dual-domain antibodies, dual variable domain (DVD) or triple variable domain (TVD) antibodies (dual-variable domain immunoglobulins and methods for making them are described in Wu, C., et al., Nature Biotechnology, 25(11):1290-1297 (2007) and PCT International Application WO 2001/058956, the contents of each of which are herein incorporated by reference), or domain antibodies (dAbs) (e.g., such as described in Holt et al. (2014) Trends in Biotechnology 21:484-490), and including single domain antibodies sdAbs that are naturally occurring, e.g., as in cartilaginous fishes and camelid, or which are synthetic, e.g., nanobodies, VHH, or other domain structure), and functionally active epitope-binding fragments of any of the above. In particular, antibodies include immunoglobulin molecules and immunologically active fragments of immunoglobulin molecules, namely, molecules that contain an analyte-binding site. Immunoglobulin molecules can be of any type (for example, IgG, IgE, IgM, IgD, IgA, and IgY), class (for example, IgG1, IgG2, IgG3, IgG4, IgA1, and IgA2).

“Antibody fragment” as used herein refers to a portion of an intact antibody comprising the antigen-binding site or variable region. The portion does not include the constant heavy chain domains (i.e. CH2, CH3, or CH4, depending on the antibody isotype) of the Fc region of the intact antibody. Examples of antibody fragments include, but are not limited to, Fab fragments, Fab′ fragments, Fab′-SH fragments, F(ab′)₂ fragments, Fd fragments, Fv fragments, diabodies, single-chain Fv (scFv) molecules, single-chain polypeptides containing only one light chain variable domain, single-chain polypeptides containing the three CDRs of the light-chain variable domain, single-chain polypeptides containing only one heavy chain variable region, and single-chain polypeptides containing the three CDRs of the heavy chain variable region.

As used herein, the term “antigen” refers to any substance that is capable of inducing an immune response. An antigen may be an organism (e.g., a fungus), a whole cell (e.g., bacterial cell) or a colony of cells, a virus, or a portion or component thereof. Examples of antigens include, but are not limited to, microbial pathogens, bacteria, viruses, proteins, glycoproteins, lipoproteins, peptides, glycopeptides, lipopeptides, lipopolysaccharides, oligosaccharides and polysaccharides, and portions or components thereof.

“Epitope,” or “epitopes,” or “epitopes of interest” refer to a site(s) on any molecule that is recognized and can bind to a complementary site(s) on its specific binding partner. The molecule and specific binding partner are part of a specific binding pair. For example, an epitope can be on a polypeptide, a protein, a hapten, a carbohydrate antigen (such as, but not limited to, glycolipids, glycoproteins or lipopolysaccharides), or a polysaccharide. Its specific binding partner can be, but is not limited to, an antibody.

The terms “specific binding partner,” “specific binding member,” and “binding member” are used interchangeably herein and refer to one of two or more different molecules that specifically recognize the other molecule compared to substantially less recognition of other molecules.

As used herein, when an antibody or other entity (e.g., antigen binding domain) “specifically recognizes” or “specifically binds” an antigen or epitope, it preferentially recognizes the antigen in a complex mixture of proteins and/or macromolecules, and binds the antigen or epitope with affinity which is substantially higher than to other entities not displaying the antigen or epitope. In this regard, “affinity which is substantially higher” means affinity that is high enough to enable selective detection of a target antigen or epitope which is distinguished from entities using a desired assay or measurement apparatus. Typically, it means binding affinity having a binding constant (Kₐ) of at least 10⁷ M⁻¹ (e.g., >10⁷ M⁻¹, >10⁸ M⁻¹, >10⁹ M⁻¹, >10¹⁰ M⁻¹, >10¹¹ M⁻¹, >10¹² M⁻¹, >1013 M⁻¹, etc.). In certain such embodiments, an antibody is capable of binding different antigens so long as the different antigens comprise that particular epitope. In certain instances, for example, homologous proteins from different species may comprise the same epitope.

As used herein, the term “subject” refers to any human or animal (e.g., non-human primate, rodent, feline, canine, bovine, porcine, equine, caprine, etc.).

As used herein, the term “sample” is used in its broadest sense and encompass materials obtained from any source. As used herein, the term “sample” is used to refer to materials obtained from a biological source, for example, obtained from animals (including humans), and encompasses any fluids, solids, and tissues. In particular embodiments of the present disclosure, biological samples include blood and blood products such as plasma, serum and the like. However, these examples are not to be construed as limiting the types of samples that find use with the present disclosure.

As used herein, the term “antibody sample” refers to an antibody-containing composition (e.g., plasma, blood, purified antibodies, blood or plasma fractions, blood or plasma components etc.) taken from or provided by a donor (e.g., natural source) or obtained from a synthetic, recombinant, other in vitro source, or from a commercial source. The antibody sample may exhibit elevated titer of a particular antibody or set of antibodies based on the pathogenic/antigenic exposures (e.g., natural exposure or through vaccination) of the donor or the antibodies engineered to be produced in the synthetic, recombinant, or in vitro context.

As used herein, the term “an amount effective to induce an immune response” (e.g., of a composition for inducing an immune response), refers to the dosage level required (e.g., when administered to a subject) to stimulate, generate and/or elicit an immune response in the subject. An effective amount can be administered in one or more administrations (e.g., via the same or different route), applications or dosages and is not intended to be limited to a particular formulation or administration route.

As used herein, the term “under conditions such that said subject generates an immune response” refers to any qualitative or quantitative induction, generation, and/or stimulation of an immune response (e.g., innate or acquired).

As used herein, the term “immune response” refers to a response by the immune system of a subject. For example, immune responses include, but are not limited to, a detectable alteration (e.g., increase) in Toll-like receptor (TLR) activation, lymphokine (e.g., cytokine (e.g., Th1 or Th2 type cytokines) or chemokine) expression and/or secretion, macrophage activation, dendritic cell activation, T cell activation (e.g., CD4+ or CD8+ T cells), NK cell activation, and/or B cell activation (e.g., antibody generation and/or secretion). Additional examples of immune responses include binding of an immunogen (e.g., antigen (e.g., immunogenic polypeptide)) to an MHC molecule and inducing a cytotoxic T lymphocyte (“CTL”) response, inducing a B cell response (e.g., antibody production), and/or T-helper lymphocyte response, and/or a delayed type hypersensitivity (DTH) response against the antigen from which the immunogenic polypeptide is derived, expansion (e.g., growth of a population of cells) of cells of the immune system (e.g., T cells, B cells (e.g., of any stage of development (e.g., plasma cells), and increased processing and presentation of antigen by antigen presenting cells. An immune response may be to immunogens that the subject's immune system recognizes as foreign (e.g., non-self antigens from microorganisms (e.g., pathogens), or self-antigens recognized as foreign). Thus, it is to be understood that, as used herein, “immune response” refers to any type of immune response, including, but not limited to, innate immune responses (e.g., activation of Toll receptor signaling cascade) cell-mediated immune responses (e.g., responses mediated by T cells (e.g., antigen-specific T cells) and non-specific cells of the immune system) and humoral immune responses (e.g., responses mediated by B cells (e.g., via generation and secretion of antibodies into the plasma, lymph, and/or tissue fluids). The term “immune response” is meant to encompass all aspects of the capability of a subject's immune system to respond to antigens and/or immunogens (e.g., both the initial response to an immunogen (e.g., a pathogen) as well as acquired (e.g., memory) responses that are a result of an adaptive immune response).

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### 2. Dual-Affinity Ratiometric Quenching (DARQ) Bioassay

Embodiments of the present disclosure include a ratiometric bioassay method for detecting a target antibody in a sample. In accordance with these embodiments, the method includes combining an antigen coupled to a first fluorophore, an antibody-binding moiety coupled to a second fluorophore, and a sample comprising or suspected of comprising a target antibody. The first fluorophore, the antibody-binding moiety coupled to the second fluorophore, and the sample can be combined in any suitable vessel, container, tube, and the like that allows for a binding complex to form (see, e.g., FIG. 2). The method includes exposing the antigen coupled to the first fluorophore, the antibody-binding moiety coupled to the second fluorophore, and the sample to fluorescent light comprising an excitation wavelength of the first and/or the second fluorophore, and detecting fluorescence emission from the first and/or the second fluorophore. In some embodiments, the fluorescence emission from the first and the second fluorophore is reduced, and wherein the reduced fluorescence emission is proportional to the antibody concentration in the sample. In some embodiments, and as described further herein, the ratiometric bioassays of the present disclosure involve fluorescence quenching of the first and/or the second fluorophore to determine antibody concentration, rather than a FRET-based mechanism. That is, the ratiometric bioassays of the present disclosure do not rely on emission from a first fluorophore to excite a second fluorophore.

In some embodiments, the antigen coupled to the first fluorophore is any antigen that is characterized a being capable of binding to the target antibody. The antigen can be the fully characterized antigen identified as being capable of binding to the target antibody, or it can be any fragment or derivative thereof, provided the portion of the antigen (e.g., epitope) recognized by the target antibody is functionally intact. As would be readily apparent to one of ordinary skill in the art based on the present disclosure, the compositions and methods provided herein can include the use of any antigen known to bind a target antibody, and any antigen subsequently developed or identified as being capable of binding a target antibody. Antigens can be obtained through any means known in the art, including but not limited to, chemical synthesis, protein purification, and genetic and cellular engineering.

As described further herein, the antigen is coupled to a first fluorophore that can be either a high-energy fluorophore or a low-energy fluorophore. In some embodiments, the antigen can be coupled to another agent in addition to the first fluorophore, provided that the additional agent does not interfere with the ability of the fluorophore to emit fluorescence or the ability of the antigen to bind the target antibody (e.g., purification tag). As would be recognized by one of ordinary skill in the art based on the present disclosure (see, e.g., Materials and Methods), coupling a fluorophore to an antigen can be done by any means known in the art. In some embodiments, coupling an antigen to a fluorophore is referred to as functional coupling because both the fluorophore and the antigen maintain at least some degree of functionality as compared to their functionality prior to coupling.

In some embodiments, the antigen is coupled to the first fluorophore at an antigen-to-fluorophore ratio of about 1:0.2 to about 1:20. In some embodiments, the antigen is coupled to the first fluorophore at an antigen-to-fluorophore ratio of about 1:0.5 to about 1:20. In some embodiments, the antigen is coupled to the first fluorophore at an antigen-to-fluorophore ratio of about 1:1 to about 1:20. In some embodiments, the antigen is coupled to the first fluorophore at an antigen-to-fluorophore ratio of about 1:1.5 to about 1:20. In some embodiments, the antigen is coupled to the first fluorophore at an antigen-to-fluorophore ratio of about 1:2 to about 1:20. In some embodiments, the antigen is coupled to the first fluorophore at an antigen-to-fluorophore ratio of about 1:3 to about 1:20. In some embodiments, the antigen is coupled to the first fluorophore at an antigen-to-fluorophore ratio of about 1:4 to about 1:20. In some embodiments, the antigen is coupled to the first fluorophore at an antigen-to-fluorophore ratio of about 1:5 to about 1:20. In some embodiments, the antigen is coupled to the first fluorophore at an antigen-to-fluorophore ratio of about 1:10 to about 1:20. In some embodiments, the antigen is coupled to the first fluorophore at an antigen-to-fluorophore ratio of about 1:15 to about 1:20. In some embodiments, the antigen is coupled to the first fluorophore at an antigen-to-fluorophore ratio of about 1:2.0 to about 1:20. In some embodiments, the antigen is coupled to the first fluorophore at an antigen-to-fluorophore ratio of about 1:0.2 to about 1:15. In some embodiments, the antigen is coupled to the first fluorophore at an antigen-to-fluorophore ratio of about 1:0.2 to about 1:10. In some embodiments, the antigen is coupled to the first fluorophore at an antigen-to-fluorophore ratio of about 1:0.2 to about 1:5. In some embodiments, the antigen is coupled to the first fluorophore at an antigen-to-fluorophore ratio of about 1:0.2 to about 1:4. In some embodiments, the antigen is coupled to the first fluorophore at an antigen-to-fluorophore ratio of about 1:0.2 to about 1:3. In some embodiments, the antigen is coupled to the first fluorophore at an antigen-to-fluorophore ratio of about 1:0.2 to about 1:2. In some embodiments, the antigen is coupled to the first fluorophore at an antigen-to-fluorophore ratio of about 1:0.2 to about 1:1. In some embodiments, the antigen is coupled to the first fluorophore at an antigen-to-fluorophore ratio of about 1:1 to about 1:10. In some embodiments, the antigen is coupled to the first fluorophore at an antigen-to-fluorophore ratio of about 1:2 to about 1:8. In some embodiments, the antigen is coupled to the first fluorophore at an antigen-to-fluorophore ratio of about 1:3 to about 1:6.

In some embodiments, the antibody-binding moiety comprises a polypeptide capable of binding a region of the target antibody that does not comprise the antigen-binding site. In some embodiments, the antibody-binding moiety coupled to the second fluorophore includes any antibody-binding moiety that is characterized a being capable of binding to the target antibody. The antibody-binding moiety can be a fully characterized polypeptide or protein identified as being capable of binding to the target antibody, or it can be any fragment or derivative thereof, provided the portion of the antibody-binding moiety that can bind to the target antibody is functionally intact. As would be readily apparent to one of ordinary skill in the art based on the present disclosure, the compositions and methods provided herein can include the use of any antibody-binding moiety known to bind a target antibody, and any antibody-binding moiety subsequently developed or identified as being capable of binding a target antibody. For example, in some embodiments, the antibody binding moiety comprises Protein L, Protein A, or Protein G, including any fragments, variants, or derivatives thereof.

As described further herein, the antibody-binding moiety is coupled to a second fluorophore that can be either a high-energy fluorophore or a low-energy fluorophore. In some embodiments, the antibody-binding moiety can be coupled to another agent in addition to the second fluorophore, provided that the additional agent does not interfere with the ability of the fluorophore to emit fluorescence or the ability of the antibody-binding moiety to bind the target antibody (e.g., purification tag). As would be recognized by one of ordinary skill in the art based on the present disclosure (see, e.g., Materials and Methods), coupling a fluorophore to an antibody-binding moiety can be done by any means known in the art. In some embodiments, coupling an antibody-binding moiety to a fluorophore is referred to as functional coupling because both the fluorophore and the antibody-binding moiety maintain at least some degree of functionality as compared to their functionality prior to coupling.

In some embodiments, the antibody-binding moiety is coupled to the second fluorophore at an antibody-binding moiety-to-fluorophore ratio of about 1:0.2 to about 1:20. In some embodiments, the antibody-binding moiety is coupled to the second fluorophore at an antibody-binding moiety-to-fluorophore ratio of about 1:0.5 to about 1:20. In some embodiments, the antibody-binding moiety is coupled to the second fluorophore at an antibody-binding moiety-to-fluorophore ratio of about 1:1 to about 1:20. In some embodiments, the antibody-binding moiety is coupled to the second fluorophore at an antibody-binding moiety-to-fluorophore ratio of about 1:1.5 to about 1:20. In some embodiments, the antibody-binding moiety is coupled to the second fluorophore at an antibody-binding moiety-to-fluorophore ratio of about 1:2 to about 1:20. In some embodiments, the antibody-binding moiety is coupled to the second fluorophore at an antibody-binding moiety-to-fluorophore ratio of about 1:3 to about 1:20. In some embodiments, the antibody-binding moiety is coupled to the second fluorophore at an antibody-binding moiety-to-fluorophore ratio of about 1:4 to about 1:20. In some embodiments, the antibody-binding moiety is coupled to the second fluorophore at an antibody-binding moiety-to-fluorophore ratio of about 1:5 to about 1:20. In some embodiments, the antibody-binding moiety is coupled to the second fluorophore at an antibody-binding moiety-to-fluorophore ratio of about 1:10 to about 1:20. In some embodiments, the antibody-binding moiety is coupled to the second fluorophore at an antibody-binding moiety-to-fluorophore ratio of about 1:15 to about 1:20. In some embodiments, the antibody-binding moiety is coupled to the second fluorophore at an antibody-binding moiety-to-fluorophore ratio of about 1:2.0 to about 1:20. In some embodiments, the antibody-binding moiety is coupled to the second fluorophore at an antibody-binding moiety-to-fluorophore ratio of about 1:0.2 to about 1:15. In some embodiments, the antibody-binding moiety is coupled to the second fluorophore at an antibody-binding moiety-to-fluorophore ratio of about 1:0.2 to about 1:10. In some embodiments, the antibody-binding moiety is coupled to the second fluorophore at an antibody-binding moiety-to-fluorophore ratio of about 1:0.2 to about 1:5. In some embodiments, the antibody-binding moiety is coupled to the second fluorophore at an antibody-binding moiety-to-fluorophore ratio of about 1:0.2 to about 1:4. In some embodiments, the antibody-binding moiety is coupled to the second fluorophore at an antibody-binding moiety-to-fluorophore ratio of about 1:0.2 to about 1:3. In some embodiments, the antibody-binding moiety is coupled to the second fluorophore at an antibody-binding moiety-to-fluorophore ratio of about 1:0.2 to about 1:2. In some embodiments, the antibody-binding moiety is coupled to the second fluorophore at an antibody-binding moiety-to-fluorophore ratio of about 1:0.2 to about 1:1. In some embodiments, the antibody-binding moiety is coupled to the second fluorophore at an antibody-binding moiety-to-fluorophore ratio of about 1:1 to about 1:10. In some embodiments, the antibody-binding moiety is coupled to the second fluorophore at an antibody-binding moiety-to-fluorophore ratio of about 1:2 to about 1:8. In some embodiments, the antibody-binding moiety is coupled to the second fluorophore at an antibody-binding moiety-to-fluorophore ratio of about 1:3 to about 1:6.

As described further herein, the method includes exposing the antigen coupled to the first fluorophore, the antibody-binding moiety coupled to the second fluorophore, and the sample to fluorescent light comprising an excitation wavelength of the first and/or the second fluorophore, and detecting fluorescence emission from the first and/or the second fluorophore. In accordance with these embodiments, the first and second fluorophores can be either high-energy or low-energy fluorophores. In some embodiments, the antigen is coupled to a first fluorophore that is a high-energy fluorophore, and the antibody-binding moiety is coupled to a second fluorophore that is a low-energy fluorophore. In some embodiments, the antigen is coupled to a first fluorophore that is a low-energy fluorophore, and the antibody-binding moiety is coupled to a second fluorophore that is a high-energy fluorophore. As described further herein, a high-energy fluorophore (or higher-energy fluorophore) generally has an excitation spectra bathocrhomically shifted from the excitation spectra of a low-energy fluorophore (or lower-energy fluorophore). In addition, the high-energy fluorophore may have an emission spectra that is also bathocrhomically shifted from the excitation spectra of the low-energy fluorophore.

In some embodiments, the high-energy fluorophore and/or the low-energy fluorophore include any fluorophore(s) capable of binding N-hydroxysuccinimde (NHS). In some embodiments, the high-energy fluorophore is selected from the group consisting of Fluorescein, Oregon Green 488, Oregon Green 514, Rhodamine Green, Rhodamine Green-X, Eosin, 4′, 6-diamidino-2-phenylindole, Alexafluor 405, Alexafluor 350, Alexafluor 500, Alexafluor 488, Alexafluor 430, Alexafluor 514, and Alexafluor 532. In some embodiments, the low-energy fluorophore is selected from the group consisting of Rhodamine, Rhodamine B, Rhodamine Red-X, Tetramethylrhodamine, Lissamine, Texas Red and Texas Red-X, Naphthofluorescein, Carboxyrhodamine 6G, Alexafluor 555, Alexafluor 546, Alexafluor 568, Alexafluor 594, Alexafluor 610, Alexafluor 633, Alexafluor 635, Alexafluor 647, Alexafluor 660, Alexafluor 680, Alexafluor 700, and Alexafluor 750. In some embodiments, the antigen is coupled to fluorescein or Alexafluor 350. In some embodiments, the antibody-binding moiety is coupled to Rhodamine.

In some embodiments, the method further includes determining the concentration of a target antibody in a sample based on the reduction in fluorescence emission of the first and/or the second fluorophore. In some embodiments, the concentration of a target antibody in a sample is based on the reduction in fluorescence emission of the first fluorophore. In some embodiments, the concentration of a target antibody in a sample is based on the reduction in fluorescence emission of the second fluorophore. In some embodiments, the concentration of a target antibody in a sample is based on the reduction in fluorescence emission of the first fluorophore and the second fluorophore.

In some embodiments, the first fluorophore and the second fluorophore are present in the composition at a first-to-second fluorophore ratio of about 1:1 to about 1:20. In some embodiments, the first fluorophore and the second fluorophore are present in the composition at a first-to-second fluorophore ratio of about 1:1 to about 1:15. In some embodiments, the first fluorophore and the second fluorophore are present in the composition at a first-to-second fluorophore ratio of about 1:1 to about 1:10. In some embodiments, the first fluorophore and the second fluorophore are present in the composition at a first-to-second fluorophore ratio of about 1:1 to about 1:5. In some embodiments, the first fluorophore and the second fluorophore are present in the composition at a first-to-second fluorophore ratio of about 1:5 to about 1:20. In some embodiments, the first fluorophore and the second fluorophore are present in the composition at a first-to-second fluorophore ratio of about 1:10 to about 1:20. In some embodiments, the first fluorophore and the second fluorophore are present in the composition at a first-to-second fluorophore ratio of about 1:15 to about 1:20. In some embodiments, the first fluorophore and the second fluorophore are present in the composition at a first-to-second fluorophore ratio of about 1:5 to about 1:15. In some embodiments, the first fluorophore and the second fluorophore are present in the composition at a first-to-second fluorophore ratio of about 1:5 to about 1:10. In some embodiments, the first fluorophore and the second fluorophore are present in the composition at a first-to-second fluorophore ratio of about 1:10 to about 1:20. In some embodiments, the first fluorophore and the second fluorophore are present in the composition at a first-to-second fluorophore ratio of about 1:10 to about 1:15.

In some embodiments, the method further includes incubating the sample comprising or suspected of comprising the target antibody, the antigen coupled to the first fluorophore, and the antibody-binding moiety coupled to the second fluorophore for 30 minutes or less prior to measuring the fluorescent emission of the first and/or the second fluorophore. In some embodiments, the method further includes incubating the sample comprising or suspected of comprising the target antibody, the antigen coupled to the first fluorophore, and the antibody-binding moiety coupled to the second fluorophore for 20 minutes or less prior to measuring the fluorescent emission of the first and/or the second fluorophore. In some embodiments, the method further includes incubating the sample comprising or suspected of comprising the target antibody, the antigen coupled to the first fluorophore, and the antibody-binding moiety coupled to the second fluorophore for 10 minutes or less prior to measuring the fluorescent emission of the first and/or the second fluorophore. In some embodiments, the method includes incubating for about 1 minute, about 2 minutes, about 3 minutes, about 4 minutes, about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 16 minutes, about 17 minutes, about 18 minutes, about 19 minutes, about 20 minutes, about 21 minutes, about 22 minutes, about 23 minutes, about 24 minutes, about 25 minutes, about 26 minutes, about 27 minutes, about 28 minutes, about 29 minutes, or about 30 minutes. In some embodiments, the method includes incubating for over 30 minutes.

In accordance with the above embodiments, the bioassay methods and compositions of the present disclosure can be used with any sample that contains, or is suspected of containing, a target antibody. In some embodiments, sample is undiluted (e.g., sampled directly) prior to the bioassay being performed. In some embodiments, sample is diluted prior to the bioassay being performed, such as with a suitable buffer or other agent. In some embodiments, the sample is obtained from an organism or a portion of an organism. In some embodiments, the sample is obtained from a bodily fluid of a mammal (e.g., a human). In some embodiments, the sample is a whole blood sample, a plasma sample, a serum sample, a urine sample, a saliva sample, or a tissue sample. In some embodiments, the sample is obtained from an industrial biological, chemical, or biochemical process. For example, in some embodiments, the sample is a cell culture sample, a cell lysate sample, or a cell culture media sample. As would be recognized by one of ordinary skill in the art based on the present disclosure, other samples may also be used with the bioassays of the present disclosure.

### 3. Compositions and Kits

Embodiments of the present disclosure also include a composition for performing a ratiometric bioassay to detect an antibody in a sample, as described above. In accordance with these embodiments, the composition includes an antigen coupled to a first fluorophore, an antibody-binding moiety coupled to a second fluorophore, and a sample comprising or suspected of comprising a target antibody. The first fluorophore, the antibody-binding moiety coupled to the second fluorophore, and the sample can be combined in any suitable vessel, container, tube, and the like that allows for a binding complex to form (see, e.g., FIG. 2). In some embodiments, fluorescence emission from the first and the second fluorophore is reduced upon exposure to fluorescent light comprising an excitation wavelength of the first and/or the second fluorophore. In some embodiments, the reduced fluorescence emission is proportional to the antibody concentration in the sample.

In some embodiments, the antibody-binding moiety comprises a polypeptide capable of binding a region of the target antibody that does not comprise the antigen-binding site. portion of the antibody-binding moiety that can bind to the target antibody is functionally intact. As would be readily apparent to one of ordinary skill in the art based on the present disclosure, the compositions and methods provided herein can include the use of any antibody-binding moiety known to bind a target antibody, and any antibody-binding moiety subsequently developed or identified as being capable of binding a target antibody. For example, in some embodiments, the antibody binding moiety comprises Protein L, Protein A, or Protein G, including any fragments, variants, or derivatives thereof.

In some embodiments, the antibody-binding moiety in the composition is coupled to a second fluorophore that can be either a high-energy fluorophore or a low-energy fluorophore. In some embodiments, the antibody-binding moiety can be coupled to another agent in addition to the second fluorophore, provided that the additional agent does not interfere with the ability of the fluorophore to emit fluorescence or the ability of the antibody-binding moiety to bind the target antibody (e.g., purification tag). As would be recognized by one of ordinary skill in the art based on the present disclosure (see, e.g., Materials and Methods), coupling a fluorophore to an antibody-binding moiety can be done by any means known in the art. In some embodiments, coupling an antibody-binding moiety to a fluorophore is referred to as functional coupling because both the fluorophore and the antibody-binding moiety maintain at least some degree of functionality as compared to their functionality prior to coupling.

In some embodiments, the antibody-binding moiety in the composition is coupled to the second fluorophore at an antibody-binding moiety-to-fluorophore ratio of about 1:0.2 to about 1:20. In some embodiments, the antibody-binding moiety is coupled to the second fluorophore at an antibody-binding moiety-to-fluorophore ratio of about 1:0.5 to about 1:20. In some embodiments, the antibody-binding moiety is coupled to the second fluorophore at an antibody-binding moiety-to-fluorophore ratio of about 1:1 to about 1:20. In some embodiments, the antibody-binding moiety is coupled to the second fluorophore at an antibody-binding moiety-to-fluorophore ratio of about 1:1.5 to about 1:20. In some embodiments, the antibody-binding moiety is coupled to the second fluorophore at an antibody-binding moiety-to-fluorophore ratio of about 1:2 to about 1:20. In some embodiments, the antibody-binding moiety is coupled to the second fluorophore at an antibody-binding moiety-to-fluorophore ratio of about 1:3 to about 1:20. In some embodiments, the antibody-binding moiety is coupled to the second fluorophore at an antibody-binding moiety-to-fluorophore ratio of about 1:4 to about 1:20. In some embodiments, the antibody-binding moiety is coupled to the second fluorophore at an antibody-binding moiety-to-fluorophore ratio of about 1:5 to about 1:20. In some embodiments, the antibody-binding moiety is coupled to the second fluorophore at an antibody-binding moiety-to-fluorophore ratio of about 1:10 to about 1:20. In some embodiments, the antibody-binding moiety is coupled to the second fluorophore at an antibody-binding moiety-to-fluorophore ratio of about 1:15 to about 1:20. In some embodiments, the antibody-binding moiety is coupled to the second fluorophore at an antibody-binding moiety-to-fluorophore ratio of about 1:2.0 to about 1:20. In some embodiments, the antibody-binding moiety is coupled to the second fluorophore at an antibody-binding moiety-to-fluorophore ratio of about 1:0.2 to about 1:15. In some embodiments, the antibody-binding moiety is coupled to the second fluorophore at an antibody-binding moiety-to-fluorophore ratio of about 1:0.2 to about 1:10. In some embodiments, the antibody-binding moiety is coupled to the second fluorophore at an antibody-binding moiety-to-fluorophore ratio of about 1:0.2 to about 1:5. In some embodiments, the antibody-binding moiety is coupled to the second fluorophore at an antibody-binding moiety-to-fluorophore ratio of about 1:0.2 to about 1:4. In some embodiments, the antibody-binding moiety is coupled to the second fluorophore at an antibody-binding moiety-to-fluorophore ratio of about 1:0.2 to about 1:3. In some embodiments, the antibody-binding moiety is coupled to the second fluorophore at an antibody-binding moiety-to-fluorophore ratio of about 1:0.2 to about 1:2. In some embodiments, the antibody-binding moiety is coupled to the second fluorophore at an antibody-binding moiety-to-fluorophore ratio of about 1:0.2 to about 1:1. In some embodiments, the antibody-binding moiety is coupled to the second fluorophore at an antibody-binding moiety-to-fluorophore ratio of about 1:1 to about 1:10. In some embodiments, the antibody-binding moiety is coupled to the second fluorophore at an antibody-binding moiety-to-fluorophore ratio of about 1:2 to about 1:8. In some embodiments, the antibody-binding moiety is coupled to the second fluorophore at an antibody-binding moiety-to-fluorophore ratio of about 1:3 to about 1:6.

In some embodiments of the composition, the first and second fluorophores can be either high-energy or low-energy fluorophores. In some embodiments of the composition, the antigen is coupled to a first fluorophore that is a high-energy fluorophore, and the antibody-binding moiety is coupled to a second fluorophore that is a low-energy fluorophore. In some embodiments of the composition, the antigen is coupled to a first fluorophore that is a low-energy fluorophore, and the antibody-binding moiety is coupled to a second fluorophore that is a high-energy fluorophore. In some embodiments, a high-energy fluorophore (or higher-energy fluorophore) generally has an excitation spectra bathocrhomically shifted from the excitation spectra of a low-energy fluorophore (or lower-energy fluorophore). In addition, the high-energy fluorophore may have an emission spectra that is also bathocrhomically shifted from the excitation spectra of the low-energy fluorophore.

In some embodiments of the composition, the high-energy fluorophore is selected from the group consisting of Fluorescein, Oregon Green 488, Oregon Green 514, Rhodamine Green, Rhodamine Green-X, Eosin, 4′, 6-diamidino-2-phenylindole, Alexafluor 405, Alexafluor 350, Alexafluor 500, Alexafluor 488, Alexafluor 430, Alexafluor 514, and Alexafluor 532. In some embodiments, the low-energy fluorophore is selected from the group consisting of Rhodamine, Rhodamine B, Rhodamine Red-X, Tetramethylrhodamine, Lissamine, Texas Red and Texas Red-X, Naphthofluorescein, Carboxyrhodamine 6G, Alexafluor 555, Alexafluor 546, Alexafluor 568, Alexafluor 594, Alexafluor 610, Alexafluor 633, Alexafluor 635, Alexafluor 647, Alexafluor 660, Alexafluor 680, Alexafluor 700, and Alexafluor 750. In some embodiments, the antigen is coupled to fluorescein or Alexafluor 350. In some embodiments, the antibody-binding moiety is coupled to Rhodamine.

In some embodiments, the bioassay methods and compositions of the present disclosure can be used with any sample that contains, or is suspected of containing, a target antibody. In some embodiments, sample is undiluted (e.g., sampled directly) prior to the bioassay being performed. In some embodiments, sample is diluted prior to the bioassay being performed, such as with a suitable buffer or other agent. In some embodiments, the sample is obtained from an organism or a portion of an organism. In some embodiments, the sample is obtained from a bodily fluid of a mammal (e.g., a human). In some embodiments, the sample is a whole blood sample, a plasma sample, a serum sample, a urine sample, a saliva sample, or a tissue sample. In some embodiments, the sample is obtained from an industrial biological, chemical, or biochemical process. For example, in some embodiments, the sample is a cell culture sample, a cell lysate sample, or a cell culture media sample. As would be recognized by one of ordinary skill in the art based on the present disclosure, other samples may also be used with the bioassays of the present disclosure.

In some embodiments, a sample refers to any fluid sample containing or suspected of containing a target antibody The sample may be derived from any suitable source. In some cases, the sample may comprise a liquid, fluent particulate solid, or fluid suspension of solid particles. In some cases, the sample may be processed prior to the analysis described herein. For example, the sample may be separated or purified from its source prior to analysis; however, in certain embodiments, an unprocessed sample containing at least one target antibody may be assayed directly. In a particular example, the source of a target antibody is a mammalian (e.g., human) bodily substance (e.g., bodily fluid, blood such as whole blood (including, for example, capillary blood, venous blood, etc.), serum, plasma, urine, saliva, sweat, sputum, semen, mucus, lacrimal fluid, lymph fluid, amniotic fluid, interstitial fluid, lower respiratory specimens such as, but not limited to, sputum, endotracheal aspirate or bronchoalveolar lavage, cerebrospinal fluid, feces, tissue, organ, one or more dried blood spots, or the like). Tissues may include, but are not limited to oropharyngeal specimens, nasopharyngeal specimens, skeletal muscle tissue, liver tissue, lung tissue, kidney tissue, myocardial tissue, brain tissue, bone marrow, cervix tissue, skin, etc. The sample may be a liquid sample or a liquid extract of a solid sample. In certain cases, the source of the sample may be an organ or tissue, such as a biopsy sample, which may be solubilized by tissue disintegration/cell lysis. Additionally, the sample can be a nasopharyngeal or oropharyngeal sample obtained using one or more swabs that, once obtained, is placed in a sterile tube containing a virus transport media (VTM) or universal transport media (UTM), for testing.

A wide range of volumes of the fluid sample may be analyzed. In a few exemplary embodiments, the sample volume may be about 0.5 nL, about 1 nL, about 3 nL, about 0.01 μL, about 0.1 μL, about 1 μL, about 5 μL, about 10 μL, about 100 μL, about 1 mL, about 5 mL, about 10 mL, or the like. In some cases, the volume of the fluid sample is between about 0.01 μL and about 10 mL, between about 0.01 μL and about 1 mL, between about 0.01 μL and about 100 μL, or between about 0.1 μL and about 10 μL. In some cases, the fluid sample may be diluted prior to use in an assay. For example, in embodiments where the source containing a target antibody is a human body fluid (e.g., blood, serum), the fluid may be diluted with an appropriate solvent (e.g., a buffer such as PBS buffer). A fluid sample may be diluted about 1-fold, about 2-fold, about 3-fold, about 4-fold, about 5-fold, about 6-fold, about 10-fold, about 100-fold, or greater, prior to use. In other cases, the fluid sample is not diluted prior to use in an assay.

Embodiments of the present disclosure also include a kit for performing a ratiometric bioassay to detect a target antibody in a sample. In accordance with these embodiments, the kit includes an antigen coupled to a first fluorophore, an antibody binding moiety coupled to a second fluorophore, and at least one container. In some embodiments, the kit further comprises a buffer and/or instructions for performing the bioassay. In some embodiments, the kit further comprises the target antibody or a fragment or derivative thereof.

In some embodiments, the kit comprises at least one component for assaying the test sample for a target antibody and corresponding instructions. Instructions included in kits can be affixed to packaging material or can be included as a package insert. While the instructions are typically written or printed materials they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” can include the address of an internet site that provides the instructions.

The kit can also comprise a calibrator or control (e.g., purified, and optionally lyophilized, target antibody or fragment thereof) and/or at least one container (e.g., tube, microtiter plates) for conducting the assay, and/or a buffer, such as an assay buffer or a wash buffer, either one of which can be provided as a concentrated solution, a substrate solution for the detectable label, or a stop solution. In some embodiments, the kit comprises all components that are necessary to perform the assay (e.g., reagents, standards, buffers, diluents, and the like). The instructions also can include instructions for generating a standard curve.

The kit may further comprise reference standards for quantifying a target antibody. The reference standards may be employed to establish standard curves for interpolation and/or extrapolation of antibody concentrations. Standards cans include proteins or peptide fragments composed of amino acids residues or labeled proteins or peptide fragments for various analytes, as well as standards for sample processing, including standards involving spikes in proteins and quantitative peptides. Kits can also include quality control components (for example, sensitivity panels, calibrators, and positive controls). Preparation of quality control reagents is well-known in the art and is described on insert sheets for a variety of products. Sensitivity panel members can be used to establish assay performance characteristics, and can be useful indicators of the integrity of bioassay kit reagents, and the standardization of assays. The kit can also include other reagents required to conduct a bioassay or facilitate quality control evaluations, such as buffers, salts, enzymes, enzyme co-factors, substrates, detection reagents, and the like. Other components, such as buffers and solutions for the isolation and/or treatment of a test sample (e.g., pretreatment reagents), also can be included in the kit.

As would be recognized and understood by one of ordinary skill in the art based on the present disclosure, the various embodiments of the ratiometric bioassay methods and compositions described herein can be used to detect antibodies specific for any antigen, such as an antigen from a pathogenic organism. In some embodiments, the ratiometric bioassay methods and compositions of the present disclosure can be used to detect and/or quantify antibodies in a sample from a subject that has been infected with a pathogenic organism or from a recombinant system that has been engineered to express such antigen-targeting antibodies. Pathogenic organisms include, but are not limited to, fungi, bacteria, viruses, and parasites. In some embodiments, detecting and/or quantifying antibodies specific for one or more antigens of a pathogenic organism indicate that the pathogenic organism has elicited an immune response in the subject. In some embodiments, recombinant systems that have been engineered to express antigen-targeting antibodies include mammalian cells (e.g., CHO and HEK293 cells) or bacteria (e.g., E. coli or Pichia pastoris or Saccharomyces cerevisiae).

In some embodiments, the ratiometric bioassay methods and compositions of the present disclosure can be used to detect antibodies in a sample from a patient (e.g., whole blood sample, a plasma sample, a serum sample, a urine sample, a saliva sample, a tissue sample, and the like) that has been infected with, or is suspected of being infected with, a coronavirus (e.g., coronavirus OC43, coronavirus 229E, coronavirus NL63, coronavirus HKU1, MERS-CoV, SARS-CoV, or SARS-CoV-2 (COVID-19)). Currently, most available serological tests that detect SARS-CoV-2 antibodies are lateral flow assays (LFA) that are based on simple positive or negative detection of antibodies, which is feasible for inexpensive and point-of-care (POC) use and large-scale surveillance but not informative regarding the amount, type, or function of the antibodies. An alternative for accurately detecting antibodies against SARS-CoV-2 is the enzyme-linked immunosorbent assay (ELISA), which can measure not only the presence but also the titer (amount) and type (IgG, IgM, monomeric or dimeric IgA) of antibody. ELISA assays allow for a better measure of the strength of the humoral response, but are complex and can only be performed in a laboratory setting. Additionally, ELISA is not ideal for virus neutralization/blocking tests, which is crucially important in studying the humoral response during vaccine development and vaccination but not widely available. Current neutralization assays usually involve propagation of viruses and require such assays to be conducted in a biosafety level 3 (BSL3) lab settings, which unfortunately is unavailable to many researchers or the public.

In some embodiments, the ratiometric bioassay methods and compositions of the present disclosure can be used to detect antibodies in a sample from a patient (e.g., whole blood sample, a plasma sample, a serum sample, a urine sample, a saliva sample, a tissue sample, and the like) that has been infected with, or is suspected of being infected with, a coronavirus (e.g., coronavirus OC43, coronavirus 229E, coronavirus NL63, coronavirus HKU1, MERS-CoV, SARS-CoV, or SARS-CoV-2 (COVID-19)). Currently, most available serological tests that detect SARS-CoV-2 antibodies are lateral flow assays (LFA) that are based on simple positive or negative detection of antibodies, which is feasible for inexpensive and point-of-care (POC) use and large-scale surveillance but not informative regarding the amount, type, or function of the antibodies. An alternative for accurately detecting antibodies against SARS-CoV-2 is the enzyme-linked immunosorbent assay (ELISA), which can measure not only the presence but also the titer (amount) and type (IgG, IgM, monomeric or dimeric IgA) of antibody. ELISA assays allow for a better measure of the strength of the humoral response, but are complex and can only be performed in a laboratory setting. Additionally, ELISA is not ideal for virus neutralization/blocking tests, which is crucially important in studying the humoral response during vaccine development and vaccination but not widely available. Current neutralization assays usually involve propagation of viruses and require such assays to be conducted in a biosafety level 3 (BSL3) lab settings, which unfortunately is unavailable to many researchers or the public.

Further, in accordance with the ratiometric bioassay methods and compositions described herein, embodiments of the present disclosure include detecting and/or quantifying coronavirus-neutralizing monoclonal antibodies in a sample obtained from a bioreactor wherein cells recombinantly engineered to express that monoclonal antibody have been cultured and produced said antibody, either as an intracellular or extracellular product. Currently, analytical assays utilized in the biopharmaceutical and biomanufacturing industries for the quantification of recombinant monoclonal antibodies rely on ELISA tests. The slow kinetics of ELISAs make them inherently off-line assays, thereby reducing the ability of optimizing the process in real time.

Neutralizing antibodies identified using the disclosed methods and compositions can specifically bind to any known or as yet undiscovered coronavirus, such as, for example, coronavirus OC43, coronavirus 229E, coronavirus NL63, coronavirus HKU1, MERS-CoV, SARS-CoV, or SARS-CoV-2 (COVID-19). In some embodiments, the neutralizing antibodies are directed against SARS-CoV-2 (COVID-19). In the context of the present disclosure a “neutralizing antibody” is an antibody that binds to a virus (e.g., a coronavirus) and interferes with the virus' ability to infect a host cell. Coronavirus spike proteins are known to elicit potent neutralizing-antibody and T-cell responses. The ability of a virus (e.g., coronavirus OC43, coronavirus 229E, coronavirus NL63, coronavirus HKU1, MERS-CoV, SARS-CoV, or SARS-CoV-2 (COVID-19)) to gain entry into cells and establish infection is mediated by the interactions of its Spike glycoproteins with human cell surface receptors. In the case of coronaviruses, Spike proteins are large type I transmembrane protein trimers that protrude from the surface of coronavirus virions. Each Spike protein comprises a large ectodomain (comprising S1 and S2), a transmembrane anchor, and a short intracellular tail. The S1 subunit of the ectodomain mediates binding of the virion to host cell-surface receptors through its receptor-binding domain (RBD). The S2 subunit fuses with both host and viral membranes, by undergoing structural changes.

SARS-CoV-2 utilizes the Spike glycoprotein to interact with cellular receptor ACE2. The amino acid sequence of the SARS-CoV-2 spike protein has been deposited with the National Center for Biotechnology Information (NCBI) under Accession No. QHD43416. Binding with ACE2 triggers a cascade of cell membrane fusion events for viral entry. The high-resolution structure of SARSCoV-2 RBD bound to the N-terminal peptidase domain of ACE2 has recently been determined, and the overall ACE2-binding mechanism is virtually the same between SARS-CoV-2 and SARS-CoV RBDs, indicating convergent ACE2-binding evolution between these two viruses. This indicates that disruption of the RBD and ACE2 interaction (e.g., by neutralizing antibodies) acts to block SARS-CoV-2 entry into the target cell. In addition, neutralizing antibodies directed against coronaviruses have been identified and isolated (see, e.g., Liu et al., Potent neutralizing antibodies directed to multiple epitopes on SARS-CoV-2 spike. Nature (2020). doi.org/10.1038/s41586-020-2571-7; Rogers et al., Science 15 Jun. 2020:eabc7520; DOI: 10.1126/science.abc7520; Alsoussi et al., J Immunol Jun. 26, 2020, ji2000583; DOI: /doi.org/10.4049/jimmunol.2000583; Kreer et al., Cell, S0092-8674(20)30821-7. 13 Jul. 2020, doi:10.1016/j.cell.2020.06.044; Tai et al., J Virol. 2017 Jan. 1; 91(1): e01651-16; and Niu et al., J Infect Dis. 2018 Oct. 15; 218(8): 1249-1260).

In accordance with the above, the ratiometric bioassay methods and compositions described herein can include the use of a coronavirus antigen, such as an antigen from the RBD of the Spike protein of SARS-CoV-2, in combination with an antibody-binding moiety (e.g., Protein L, Protein A, or Protein G). In some embodiments, the coronavirus antigen is coupled to a first fluorophore, and the antibody-binding moiety is coupled to a second fluorophore, wherein the fluorescence emission from the first and the second fluorophore is reduced, and wherein the reduced fluorescence emission is proportional to the antibody concentration in the sample, as described further herein. In this manner, neutralizing antibodies directed against SARS-CoV-2 can be detected and/or quantified in a sample from a patient or from a recombinant source. In some embodiments, the coronavirus antigen (e.g., peptide comprising a rRBD of a coronavirus spike protein) may be prepared using routine molecular biology techniques, as would be recognized by one of ordinary skill in the art based on the present disclosure. The nucleic acid and amino acid sequences of RBDs of various coronavirus spike proteins are known in the art (see, e.g., Tai et al., Cell Mol Immunol 17, 613-620 (2020). doi.org/10.1038/s41423-020-0400-4; and Chakraborti et al., Virology Journal volume 2, Article number: 73 (2005); and Chen et al., Biochemical and Biophysical Research Communications, 525(1): 135-140 (2020)). An exemplary RBD domain of a SARS-CoV-2 spike protein can comprise the following amino acid sequence:

RVQPTESIVRFPNITNLCPFGEVFNATRFASVYAWNRKRISNCVADYSV
LYNSASFSTFKYGVSPTKLNDLCFTNVYADSFVIRGDEVRQIAPGQTGK
IADYNYKLPDDFTGCVIAWNSNNLDSKVGGNYNYLYRLFRKSNLKPFER
DISTEIYQAGSTPCNGVEGFNCYFPLQSYGFQPTNGVGYQPYRVVVLSF
ELLHAPATVCGPKKSTNLVKNKCVNFNFNGLTGTGVLTESNKKFLPFQQ
FGRDIADTTDAVRDPQTLEILDITPCS.

In some embodiments, the ratiometric bioassay methods and compositions described herein can be used to assess the coronavirus neutralization capacity or titer of a sample from an individual subject, or the coronavirus neutralization capacity or titer of a sample from multiple subjects (e.g., pooled plasma samples) or the coronavirus neutralization capacity or titer of a sample from a bioreactor (e.g., a cell culture fluid obtained from cells engineered to express the coronavirus neutralizing antibody). In some embodiments, the ratiometric bioassay methods and compositions described herein can be used to determine whether a subject has been infected with a coronavirus. In some embodiments, the ratiometric bioassay methods and compositions described herein can be used to determine whether a subject that has been vaccinated against a coronavirus has elicited a sufficient immune response to the vaccine (e.g., produced sufficiently neutralizing coronavirus antibody titers). In some embodiments, the ratiometric bioassay methods and compositions described herein can be used to determine whether the engineered cells have expressed (either intracellularly or extracellularly) the coronavirus-targeting antibody.

### 4. Materials and Methods

Materials.

Pierce recombinant Protein L (PrL), NHS-Rhodamine, NHS-Fluorescein, and Coomassie (Bradford) protein assay were from Thermo Scientific (Waltham, MA, USA). Human TNF-α from Shenandoah Biotechnology Inc. (Warwick, PA, USA), while Her2 was sourced from ACRObiosystems (Newark, DE, USA). Anti-TNF-α hIgG1 (Adalimumab) was sourced from InvivoGen (San Diego, CA, USA), while anti-Her2 hIgG1 (Trastuzumab) was obtained from Selleckchem (Houston, TX, USA). Phosphate Buffer Saline at pH 7.4 (PBS), sodium bicarbonate, sodium hydroxide, aqueous HCl, dimethylsulfoxide (DMSO) and Amicon Ultra centrifugal filters (0.5 mL, 3KDa MWCO) from Millipore Sigma (St. Louis, MO, USA). 96 well non-binding microplates (F-Bottom/Chimney Well, Solid, Black) were from Greiner Bio-One (Monroe, NC, USA), while the 96 well non-treated microplates were from Thermo Fisher Scientific (Waltham, MA, USA). The null Chinese Hamster Ovary (CHO-S) cell culture fluid was donated by the Biomanufacturing Training and Education Center (BTEC) at NC State University.

Protein Labeling and Quantification.

NHS-Fluorescein and NHS-Rhodamine were initially dissolved in DMSO at 5 mg·mL⁻¹ and 10 mg·mL⁻¹, respectively. PrL, Her2, and TNF-α were initially dissolved in 50 mM sodium bicarbonate at pH 8.5 at 10 mg·mL⁻¹. A volume of 5.4 μL of NHS-Fluorescein solution in DMSO and 50 μL of antigen (either Her2 or TNF-α) solution were incubated at 0° C. for 2 hours in dark. A volume of 18 μL of NHS-Rhodamine solution in DMSO and 50 μL of PrL solution were incubated at 0° C. for 2 hours in dark. Following incubation, the fluorescent labels were removed by centrifugation with Amicon Ultra centrifugal filters (0.5 mL, 3KDa MWCO) at 14,000×g for 20 min, followed by diafiltration with PBS at pH 7.4. The concentration of ^{Rh}PrL, ^{Fl}Her2, and ^{Fl}TNF-α in PBS was determined by Bradford assay, following manufacturer's specifications. The absorbance of the solutions of ^{R}PrL and fluorescein-labeled antigen (^{Fl}Her2 and ^{Fl}TNF-α) were measured by UV spectrophotometry at a wavelength of 494 nm and 552 nm, respectively, using a Synergy H1 plate reader (Biotek, Winooski, VT). From these values, the values of fluorophore-to-protein (F/P) ratio for the various labeled proteins were calculated using Equation 1.

$$Moles dye per mole protein = \frac{{A}_{max} of labeled protein}{{ε}^{′} \times protein concentration ( M )} \times dilution factor \qquad ( 1 )$$

Quantification of Trastuzumab and Adalimumab Via DARQ Assay in Non-Competitive Conditions.

A volume of 500 μL of 57.4 μg·mL⁻¹ fluorescein-labeled antigen (^{Fl}Her2 or ^{Fl}TNF-α) and 500 μL of 20 μg·mL^{-1 Rh}PrL were mixed with 11 mL of PBS to reach a total volume of 12 mL at 25.2 nM of each protein. A volume of 230 μL of ^{R}PrL/^{Fl}Her2 mixture was dispensed in 48 wells of a 96 well non-binding microplate, and 20 μL of solution of Trastuzumab at different concentrations was added to every well to achieve a final concentration of 23.2 nM for both ^{Rh}PrL and ^{Fl}Her2, and a concentration of Trastuzumab varying between 0.92 and 18.4 nM. Additional sets of samples serving as negative control were prepared by replacing either ^{Fl}Her2 or ^{Rh}PrL with a corresponding volume of PBS. Upon mixing the mAb solution with the fluorescently labeled affinity tags, the solutions were incubated for either 1, 5, 10, or 20 min at room temperature, and analyzed by fluorescence spectrophotometry using a Synergy H1 plate reader operated at the excitation wavelength of 480 nm and emission wavelengths of 525 nm and 573 nm. An identical set of experiments was repeated using Adalimumab, ^{Rh}PrL, and ^{Fl}TNF-α.

Quantification of Trastuzumab and Adalimumab Via DARQ Assay in Competitive Conditions.

The experiments described herein were repeated by dissolving the lyophilized mAb, ^{Rh}PrL, and ^{Fl}Her2 in clarified cell culture harvest produced with a null (non mAb-expressing) Chinese Hamster Ovary (CHO-S) cell line.

Evaluation of the Fluorescence Quenching Mechanism.

PrL, Her2, and TNF-α were initially dissolved in 50 mM sodium bicarbonate at pH 8.5 at 10 mg·mL⁻¹; in parallel, NHS-Fluorescein, NHS-Rhodamine, and NHS-AlexaFluor 350 (AF350) were dissolved in DMSO at 5 mg·mL⁻¹, 10 mg·mL⁻¹, and 10 mg·mL⁻¹ respectively. A volume of 2.26 μL of NHS-Rhodamine solution in DMSO and 25 μL of TNF-α solution were incubated at 0° C. for 2 hours in dark; in parallel, 22.1 μL of NHS-Rhodamine solution in DMSO with 140 μL of PrL solution, and 1.80 μL of NHS-AF350 solution in DMSO with 62 μL of TNF-α solution were incubated at 0° C. for 2 hours in dark. Following incubation, the fluorescent labels were removed by diafiltration as described above. The solutions of ^{Rh}PrL, ^{P} TNF-α, and ^{AF350}TNF-α in PBS were analyzed via Bradford assay and UV spectrophotometry at the wavelength of 552 nm to determine the values of fluorophore-to-protein (F/P) ratio using Equation 1. A volume of 500 μL of ^{Rh}TNF-α solution at 57.4 μg·mL⁻¹ and 500 μL of ^{Rh}PrL solution at 20 μg·mL⁻¹ were mixed with 11 mL of PBS to produce 12 mL of ^{Rh}TNF-α/^{Rh}PrL stock solution wherein the concentration of each protein is 25.2 nM. In parallel, a volume of 500 μL of ^{AF350}TNF-α solution at 57.4 μg·mL⁻¹ and 500 μL of ^{Rh}PrL solution at 20 μg·mL⁻¹ were mixed with 11 mL of PBS to produce 12 mL of ^{AF350}TNF-α/^{Rh}PrL stock solution wherein the concentration of each protein is 25.2 nM; additional stock solutions were made with identical composition, wherein unlabeled TNF-α was used in lieu of ^{Rh}TNF-α to produce a TNF-α/^{Rh}PrL stock solution, and unlabeled PrL was used in lieu of ^{Rh}PrL to produce a ^{AF350}TNF-α/PrL stock solution.

Aliquots of 230 μL of ^{Rh}PrL/^{Rh}TNF-α stock solution were dispensed in 48 wells of a 96 well non-binding microplate, to which 20 μL aliquots of Adalimumab solutions at different concentrations were added to achieve a final concentration in each well of 23.2 nM for both ^{Rh}PrL and ^{Rh}TNF-α, and varying between 0.92 and 18.4 nM for Adalimumab. Another 48 wells were filled with samples with identical composition, with the sole difference that ^{Rh}PrL/TNF-α was used as stock solution (unlabeled TNF-α). Upon mixing, the samples were incubated for 5 min at room temperature and analyzed by fluorescence spectrophotometry at the excitation wavelength of 480 nm and emission wavelengths of 525 nm and 573 nm, along with an excitation of 525 nm and emission at 573 nm.

Aliquots of 230 μL of ^{AF350}TNF-α/^{Rh}PrL stock solution were dispensed in 48 wells of a 96 well non-binding microplate, to which 20 μL aliquots of Adalimumab solutions at different concentrations were added to achieve a final concentration in each well of 23.2 nM for both ^{AF350}TNF-α and ^{Rh}PrL, and varying between 0.92 and 17.8 nM for Adalimumab. Another 48 wells were filled with samples with identical composition, with the sole difference that ^{AF350}TNF-α/PrL was used as stock solution (unlabeled PrL). Upon mixing, the samples were incubated for 25 min at room temperature and analyzed by fluorescence spectrophotometry at the excitation wavelength of 335 nm and emission wavelengths of 445 nm and 573 nm, along with an excitation of 525 nm and emission at 573 nm.

Statistical Data Analysis.

The Student's t-test was performed to identify the Limit of Detection (i.e., statistically significantly different from a blank sample). The Wald-Wolfowitz runs test (departure from linearity) was performed to respectively determine dynamic range and sensitivity. The coefficient of variation (CoV) was calculated to evaluate reproducibility and repeatability of the tests.

It is understood that the foregoing detailed description and accompanying examples are merely illustrative and are not to be taken as limitations upon the scope of the disclosure, which is defined solely by the appended claims and their equivalents.

All publications and patents mentioned in the above specification are herein incorporated by reference as if expressly set forth herein. Various changes and modifications to the disclosed embodiments will be apparent to those skilled in the art and may be made without departing from the spirit and scope thereof.

### 5. Examples

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present disclosure has multiple aspects, illustrated by the following non-limiting examples.

### Example 1

Protein Labeling and Quantification.

The fluorescent affinity tags were initially prepared by labeling PrL with NHS-rhodamine (^{Rh}PrL), Her2 with NHS-fluorescein (^{Fl}Her2), and TNF-α with NHS-AF350, NHS-fluorescein, and NHS-rhodamine (^{AF350}TNF-α, ^{Fl}TNF-α, ^{Rh}TNF-α). The fluorophores were conjugated to the free amine groups of the lysine residues of the proteins via NHS chemistry. The resulting values of fluorophore-to-protein (F/P) ratios and rhodamine-to-fluorescein (Rh/Fl) ratios, listed in Table 1, were determined via Bradford assay and UV spectrophotometry. The rhodamine:fluorescein ratio of 8-9 adopted in this study was inspired by the report of Lichlyter et al. on a FRET-based immunosensor, where a ratio <5 resulted in the fluorescence of the donor (fluorescein) dominating the signal; whereas the donor fluorescence becomes undetectable for ratios >27. In designing the assay, it was hypothesized that by maintaining an optimized acceptor:donor ratio ˜8-9, the fluorescent affinity tags framed in the affinity complex would exhibit a ratiometric fluorescence effect that is correlated to the concentration of the target mAb.

TABLE 1
Values of fluorophore to protein (F/P), and rhodamine-to-fluorescein
(Rh/Fl) and rhodamine-to-AF350 (Rh/AF350) ratios for ^{AF350}TNF-α,
^{Fl}TNF-α, ^{Rh}TNF-α, ^{Fl}Her2, and ^{Rh}PrL.

| | | F/P Ratio | Rhodamine: | Rh/AF-350 |
|---|---|---|---|---|
| Protein | Fluorophore | (mol) | Fluorescein | Ratio |
| PrL | NHS-Rhodamine | 5.01 | 9.17 | N/A |
| Her2 | NHS-Fluorescein | 0.55 | | |
| PrL | NHS-Rhodamine | 11.9 | 7.99 | N/A |
| TNF-α | NHS-Fluorescein | 1.49 | | |
| PrL | NHS-Rhodamine | 7.32 | 13.60 | N/A |
| TNF-α | NHS-Rhodamine | 0.54 | | |
| PrL | NHS-Rhodamine | 7.32 | N/A | 20.9 |
| TNF-α | NHS-AF350 | 0.35 | | |

### Example 2

Quantification of Trastuzumab by DARQ Assay.

Large-scale manufacturing of therapeutic mAbs relies on recombinant expression by CHO cells and purification using a series of chromatographic steps. The mAb titer along the production pipeline is currently monitored via off-line assays—typically, ELISA and analytical Protein A HPLC—that suffer from laborious sample preparation, long run time, and expensive equipment and consumables. To overcome the limitations of current assays a novel, affordable, and rapid (single-step) assay was developed that can be applied at-line for accurate mAb quantification in process fractions. The assay was first demonstrated using Trastuzumab/Her2 as model mAb/AG pair. Trastuzumab, marketed as Herceptin by Genentech, is among the earliest mAbs utilized for human therapy and is currently utilized in the fight against breast and stomach cancer.

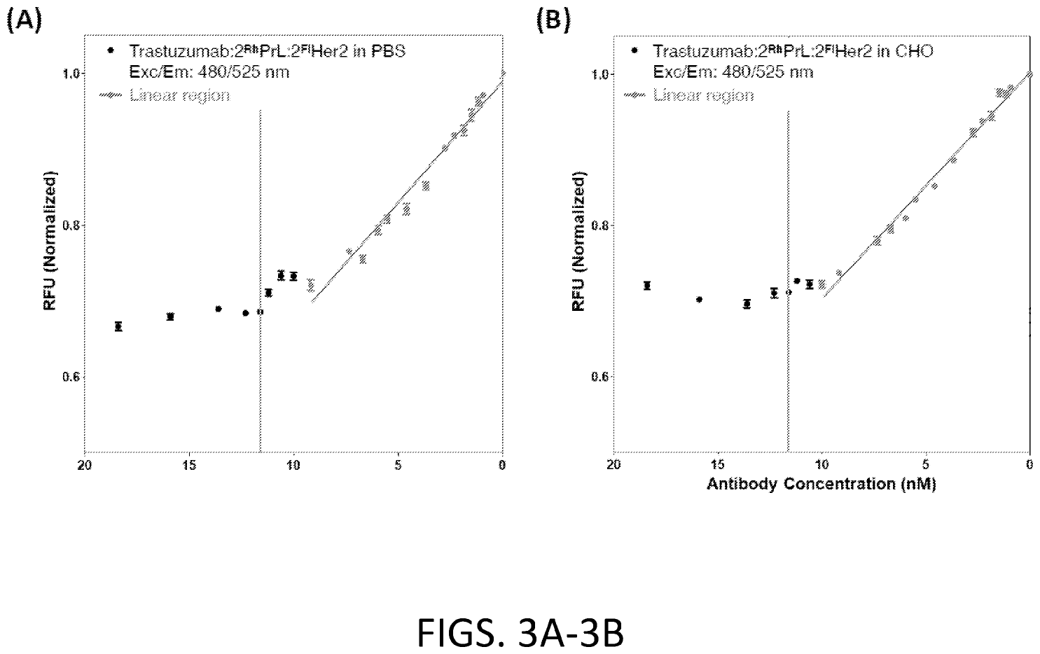

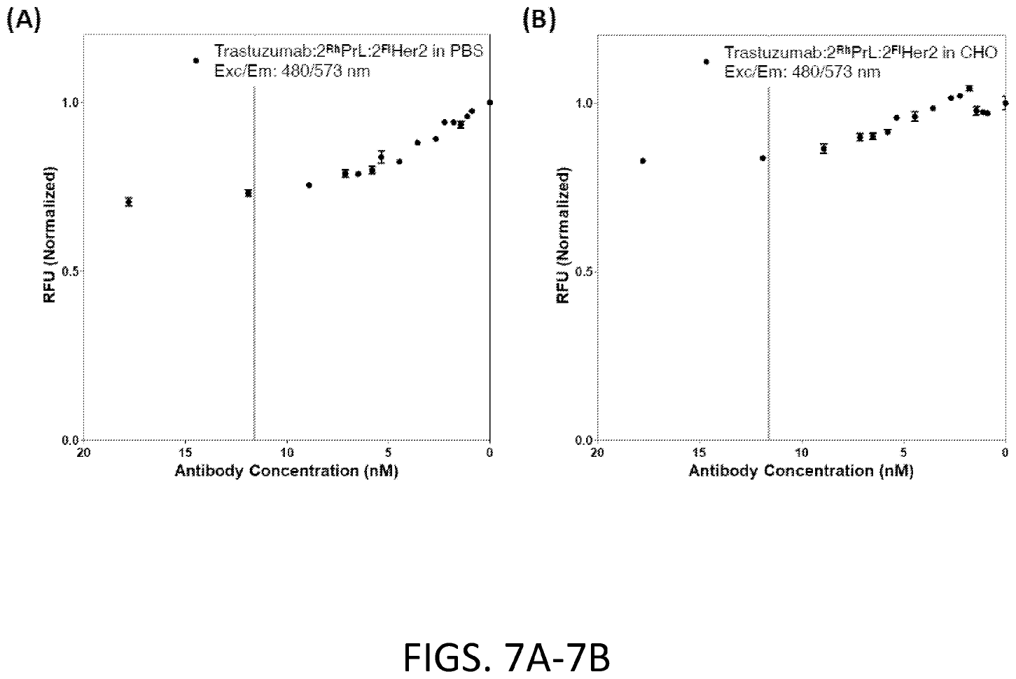

An ensemble of stock solutions of Trastuzumab were initially prepared either in PBS (non-competitive conditions) or CHO cell culture harvest (competitive conditions, CHO host cell protein titer ˜0.4 mg·mL⁻¹, which is typical of industrial cell culture fluids, and contacted with a pre-formulated ^{Rh}PrL/^{Fl}Her2 mix to achieve a final concentration of 23.2 nM for both ^{Rh}PrL and ^{Fl}Her2, and a concentration of Trastuzumab varying between 0.92 and 18.4 nM. The molar excess of ^{Rh}PrL and ^{Fl}Her2 enables the rapid formation of the mAb:^{Rh}PrL:^{Fl}Her2 affinity complex. After a short incubation time (25 min), the samples were analyzed by fluorescence spectrophotometry at λₑₓ=480 nm and λₑₘ=525 nm (green) or 573 nm (yellow). The resulting value of normalized fluorescent emission intensity vs. mAb concentration in solution are reported in FIG. 3A and FIG. 3B for non-competitive and competitive conditions, respectively. In both cases, the normalized fluorescent signal was found to decrease linearly with the antibody concentration, indicating that the framing of ^{Rh}Pr and ^{Fl}Her2 at a fixed molecular orientation and distance by Trastuzumab results in a ratiometric quenching of fluorescence emissions at the 525 nm wavelength. The values of limit of detection (LOD), dynamic range (DR), and sensitivity (S), collated in Table 2, were derived from statistical analysis of the raw data (Tables 7 and 8). Notably, no significant difference among the listed parameters was observed between competitive and non-competitive conditions—likely a result of the high affinity and selectivity of PrL and Her2 for Trastuzumab—indicating robustness of the assay. With λₑₓ=480 nm and λₑₘ=573 nm, the normalized rhodamine signal was found to decrease linearly with antibody concentration (FIG. 7), although with a smaller sensitivity when compared to the observed decrease at λₑₘ=525 nm. These results suggest that a FRET-like interaction between fluorescein and rhodamine is not realized, as it would have manifested in an increase in fluorescence emission at 573 nm concurrent with the decrease at 525 nm.

TABLE 2
Values of sensitivity (S), limit of detection (LoD), dynamic range
(DR), and coefficient of variation (CoV) for the DARQ
quantification of Trastuzumab using the dual
fluorescence affinity complex Trastuzumab:2^{Rh}PrL:2^{Fl}Her2.

| | S (ng · mL⁻¹) | LoD (ng · mL⁻¹) | DR (ng · mL⁻¹) | CoV (%) |
|---|---|---|---|---|
| Her2 in PBS | 0.54 | 160 | 531-1358 | 1.16 |
| Her2 in CHO | 0.36 | 75 | 250-1480 | 0.82 |

### Example 3

Quantification of Adalimumab (Humira) by DARQ Assay.

The assay was repeated using Adalimumab/TNF-α as the mAb/antigen pair. Adalimumab, released in the US in 2002, is listed by the World Health Organization's among the safest and most effective therapeutics for treating a number of autoimmune disorders, including rheumatoid arthritis and Crohn's disease. Like Trastuzumab, Adalimumab is a κI-IgG1 antibody and is mass manufactured via recombinant expression in engineered CHO cell lines.

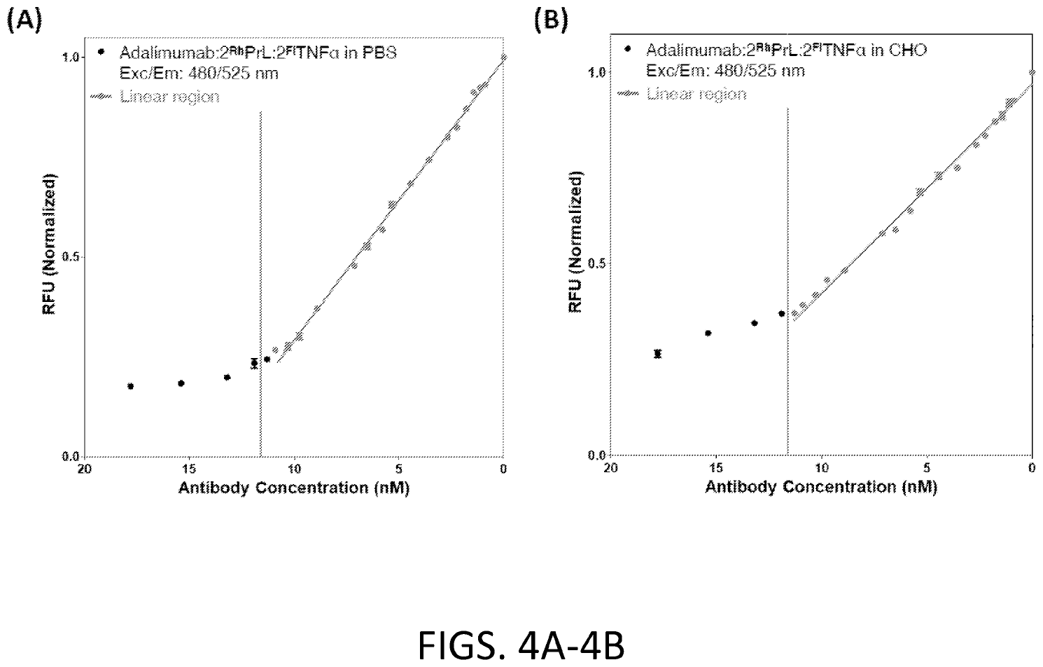

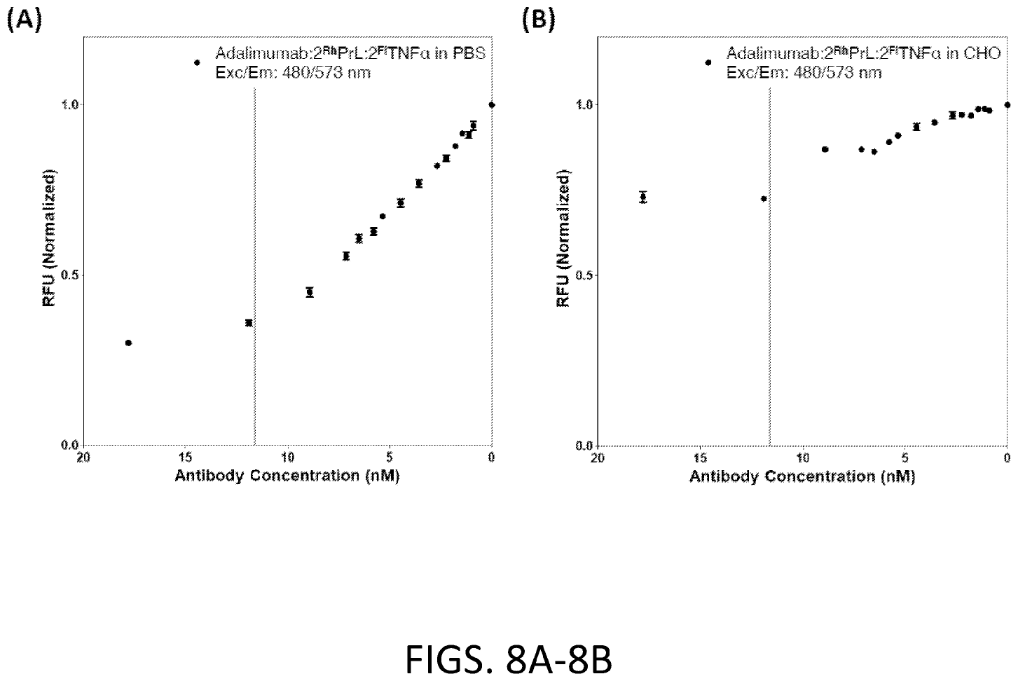

The application of the assay to the Adalimumab/TNF-α pair returned once again a linear decrease of normalized fluorescent emission intensity vs. mAb concentration under both non-competitive (FIG. 4A) and competitive conditions (FIG. 4B). The resulting values of LOD, DR, and S (Table 3), derived from statistical analysis of the raw data (Tables 9 and 10) are also on par with those obtained with Trastuzumab. The normalized fluorescent signal generated with λₑₓ=480 nm and λₑₘ=573 nm decreased linearly with antibody concentration (FIG. 8), but with a lesser sensitivity when compared to the λₑₘ=525 nm.

TABLE 3
Values of sensitivity (S), limit of detection (LoD), dynamic
range (DR), and coefficient of variation (CoV) for the DARQ
quantification of Adalimumab using the dual
fluorescence affinity complex Adalimumab:2^{Rh}PrL:2^{Fl}TNF-α.

| | S (ng · mL⁻¹) | LoD (ng · mL⁻¹) | DR (ng · mL⁻¹) | CoV (%) |
|---|---|---|---|---|
| TNF-α in PBS | 0.24 | 51 | 369-1613 | 2.76 |
| TNF-α in CHO | 0.23 | 78 | 469-1672 | 1.28 |

### Example 4

Fluorescence Quenching Mechanism.

The findings reported in FIGS. 3 and 4 were somewhat unexpected: while the choice of fluorophores and the molecular distance between the fluorescent affinity tags are characteristic of Förster Resonance Energy Transfer phenomena (e.g., FRET), the outcome resembles that of fluorescence quenching.

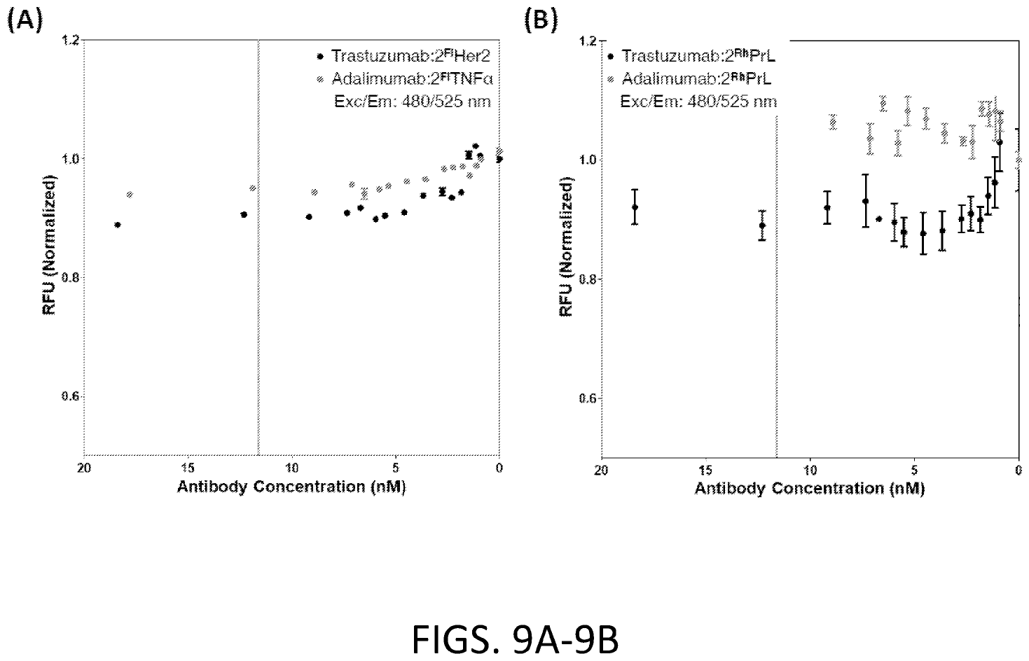

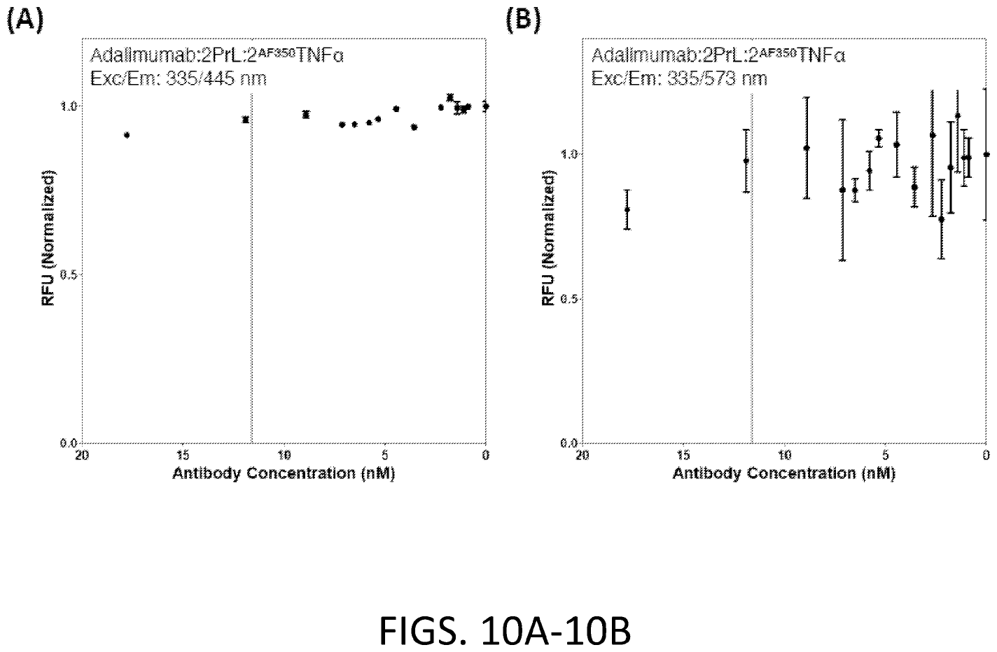

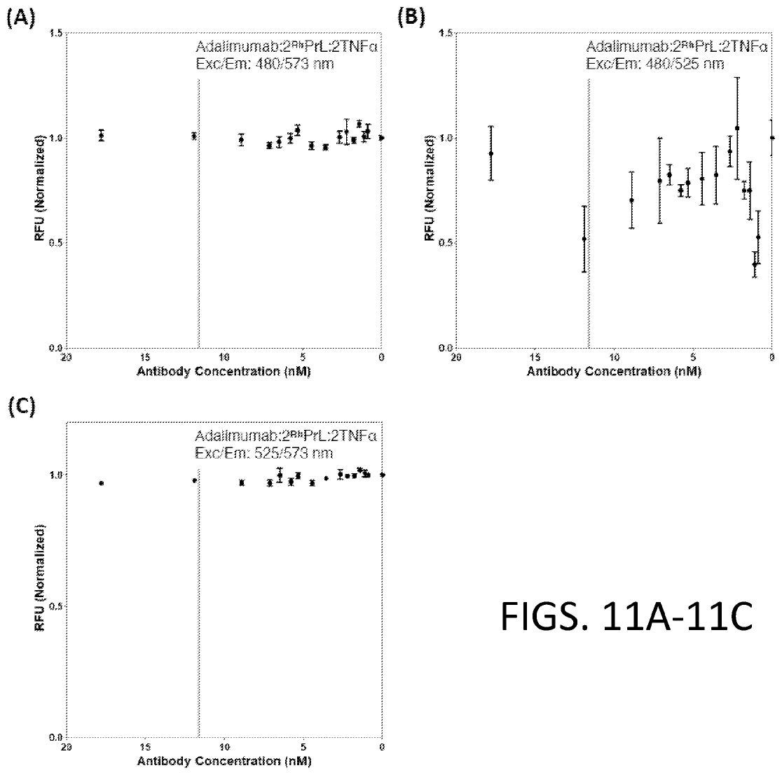

It was initially hypothesized that the results could be ascribed to amino acid-driven fluorescence quenching, which has been observed in prior studies with several organic dyes including fluorescein and rhodamine. The molecular architecture of the Adalimumab:2^{Rh}PrL:2^{Fl}TNF-α complex exemplified in FIG. 1 indeed suggests that some fluorescent dyes can be “caged” by a proteinaceous shell that, owing to the aromatic residues (e.g., tryptophan) contained therein, quenches their fluorescence emission. To evaluate this hypothesis, the fluorescence emission of the single dye—single affinity complexes Adalimumab:2^{Fl}AG and Adalimumab:2^{Rh}PrL (FIG. 9) were measured, as well as the single dye—dual affinity complexes Adalimumab:2PrL:2^{AF350}TNF-α (FIG. 10) and Adalimumab:2^{Rh}PrL:2TNF-α (FIG. 11). All four complexes, however, showed negligible fluorescence quenching with mAb concentration, indicating that protein “caging” of the fluorophores is not responsible for the observed ratiometric quenching.

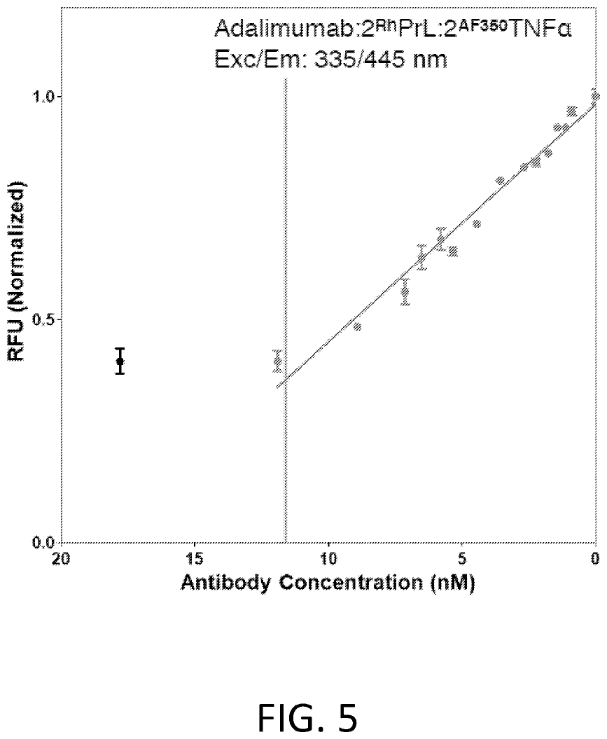

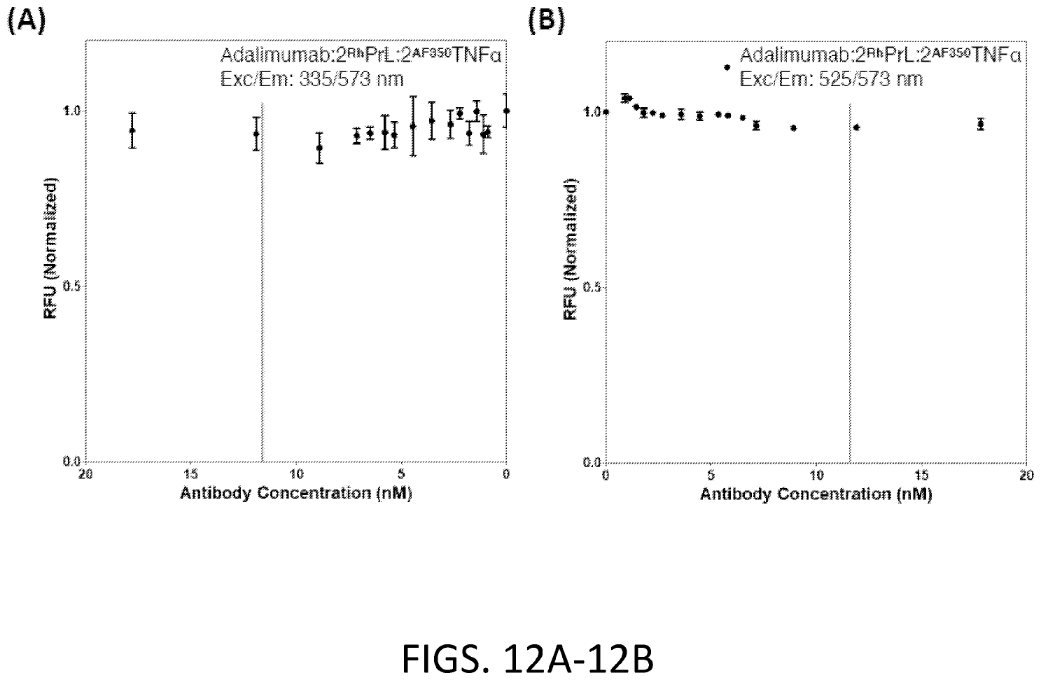

Experiments were then conducted to investigate whether the mechanism underlying the DARQ assay is of the nature of energy transfer quenching or some other immobilization based quenching effect. To this end the fluorophore AF350 (blue) was utilized in lieu of Fl. Unlike the fluorescein-rhodamine pair (J-integral_{Fl-Rh}˜5·10¹⁵ m⁻¹·cm⁻¹·nm⁴), in fact, AF350 and fluorescein do not engage in energy transfer (J-integral_{AF350-Fl}˜0). Accordingly, the fluorescence emission of the affinity complex Trastuzumab:2^{Rh}PrL:2^{AF350}Her2 was measured, and the same linear decrease in fluorescence signal was observed, as above (FIG. 5; Table 4, and FIG. 12), indicating that the DARQ assay does not operate upon energy transfer mechanisms.

TABLE 4
Values of sensitivity (S), limit of detection (LoD), dynamic
range (DR), and coefficient of variation (CoV) for the
DARQ quantification of Adalimumab using the dual
fluorescence affinity complex Adalimumab:2^{Rh}PrL:2^{AF350}TNF-α.

| | S (ng · mL⁻¹) | LoD (ng · mL⁻¹) | DR (ng · mL⁻¹) | CoV (%) |
|---|---|---|---|---|
| TNF-α in PBS | 0.54 | 233 | 527-1761 | 11.4 |

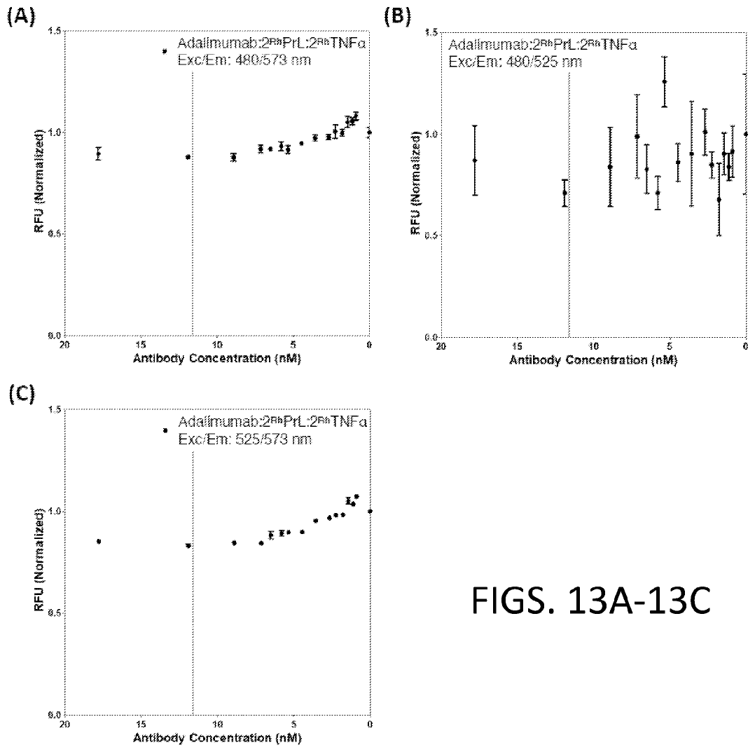

Experiments were conducted to evaluate whether other quenching phenomena occur, such as self-quenching (homo-FRET), that drive the linear decrease in fluorescence emission. To this end, the fluorescence emission of the affinity complex Adalimumab:2^{Rh}PrL:2^{Rh}TNF-α formed by Rh-only labeled affinity tags was measured. Once again, however, no fluorescence quenching was observed with mAb concentration (FIG. 13), excluding the occurrence of self-quenching (homo-FRET).

Collectively, these results suggest that the mechanism underlying the DARQ assay is a dynamic quenching mechanism. The fluorescence quenching observed in these results consistently favors the quenching of the high-energy fluorophores (i.e., AF350 and Fl), yet it requires the presence of a low-energy fluorophore (Rh); in fact, no ratiometric quenching is observed when PrL is unlabeled, as shown in FIG. 10.

Further corroborating this hypothesis is the observation that the DARQ assay featured a 2-fold higher sensitivity for Adalimumab than for Trastuzumab. This can be attributed to the substantial difference in the size of the antigens. As shown in FIG. 1D, in fact, TNF-α is a small protein (MW˜17.3 kDa, rhodamine˜3.2 nm), and the fluorescein moieties displayed on its surface are all located at short distance (˜3-4 nm) to the rhodamine moieties conjugated on PrL. Conversely, as shown in FIG. 1C, Her2 has a much higher size (MW˜88.7 kDa, rhodamine˜5.5 nm), and can carry several fluorescein moieties at a distance from ^{Rh}PrL at which dye-dye quenching does not occur.

### Example 5

Effect of Incubation Time of the mAb:^{Rh}PrL:^{Fl}AG Complex on DARQ.

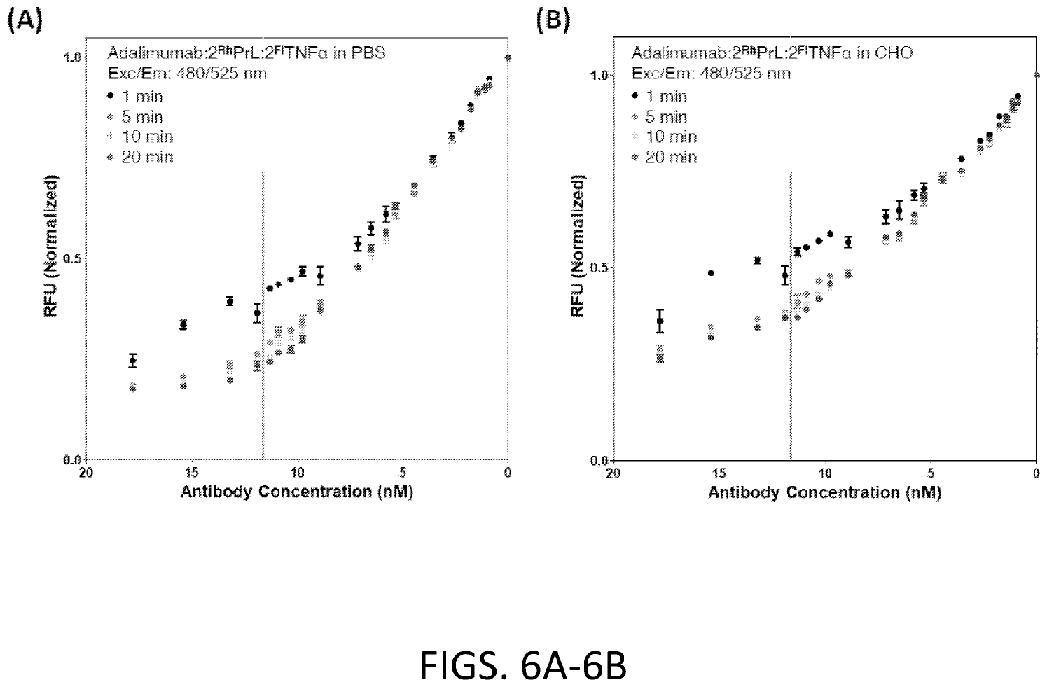

Experiments were conducted to evaluate the performance of the assay with respect to the incubation time of the target mAb with the fluorescently labeled proteins using Adalimumab/TNF-α as model mAb/antigen pair. The assay was conducted as explained above, while reducing the incubation time from 20 min to 10, 5, and 1 min. As anticipated, a linear decrease in the normalized fluorescent emission intensity vs. mAb concentration was observed for all incubation time points under both non-competitive (FIG. 6A) and competitive conditions (FIG. 6B). The resulting values of S, LOD, and DR, obtained from statistical analysis of the raw data (Tables 9-10 and Tables 11-16), are reported in Table 5. Exception made for a slight increase in the dynamic range obtained with longer incubation times (10 and 20 min), the assay performance parameters did not vary with incubation time. This is coherent with the rapid binding kinetics characteristic of mAb:PrL and mAb:antigen complexes.

TABLE 5
Values of sensitivity (S), limit of detection (LoD), dynamic
range (DR), and coefficient of variation (CoV) for the
quantification of Adalimumab via the dual fluorescent
affinity tag assay conducted at different incubation times.

| | Incubation | S | LoD | DR | CoV |
|---|---|---|---|---|---|
| | time (min) | (ng · mL⁻¹) | (ng · mL⁻¹) | (ng · mL⁻¹) | (%) |
| TNF-α in | 0 | 0.26 | 48 | 338-1318 | 2.16 |
| PBS | 5 | 0.24 | 72 | 560-1443 | 2.57 |
| | 10 | 0.24 | 39 | 312-1443 | 2.54 |
| | 20 | 0.24 | 51 | 396-1613 | 2.36 |
| TNF-α in | 0 | 0.24 | 67 | 349-1443 | 1.72 |
| CHO | 5 | 0.21 | 22 | 125-1672 | 2.05 |
| | 10 | 0.21 | 56 | 0353-1443 | 1.77 |
| | 20 | 0.23 | 78 | 469-1672 | 1.28 |

### Example 6

Comparison of DARQ with Established Analytical Methods for Antibody Quantification.

Table 6 compares the performance of the DARQ assay to that of state-of-the-art assays for the quantification of Adalimumab and Trastuzumab. DARQ performs comparably to the reference assays in terms of limit of detection and dynamic range but is considerably superior in terms of ease and speed of operation. Chromatographic assays require extensive sample preparation, a trained operator, and expensive equipment. Commercial ELISA kits are attractive owing to their low limit of detection and ample dynamic range; on the other hand, they suffer from high variability, entail a series of time-consuming steps (blocking, incubation of the sample and primary/secondary detection antibodies, intermediate washes, and enzymatic propagation of the signal), and require expensive automated liquid handlers to secure acceptable throughput. Other assays have recently been commercialized, which feature low limit of detection (˜10 ng/mL for each mAb) and a 10-to-100-fold span of dynamic range. Cardinali et al. have developed a peptide-based ELISA that performs comparably to antibody-based ELISAs while improving on throughput. The Quantum Blue Adalimumab lateral flow assay (BUHLMANN Laboratories) performs well and is rapid compared to the other testing methods; however, its applicability to industrial settings is limited by the need of a separate 15-minute test for each sample. The HMSA and UPLC methods, respectively developed by Wang et al. and Russo et al., are impressive in range and sensitivity, but require laborious sample preparation. The DARQ assay provides competitive assay parameters (sensitivity, limit of detection, and dynamic range), while dramatically reducing assay variability and time to measurement. This makes DARQ a practical choice for mAb quantification when time or equipment are limited.

TABLE 6
Survey of reported analytical methods for the quantification
of Adalimumab and Trastuzumab: limit of detection (LoD),
dynamic range (DR), time to result, and dilution
factor. Abbreviations: Homogenous Mobility Shift Assay
(HMSA), Ultra-performance liquid chromatography (UPLC),
Multiple Reaction Monitoring Mass Spectrometry (MRM-MS).

| | | LOD | DR | | |
|---|---|---|---|---|---|
| | | (ng · | (ng · | Time to | Dilution |
| Assay | Target | mL-1) | mL-1) | Run | Factor |
| DARQ | Adalimumab | 141 | 370-1,600 | ~5 min | No sample |
| | | | | | dilution |
| DARQ | Trastuzumab | 75 | 250-1,480 | ~25 min | No sample |
| | | | | | dilution |
| ELISA | Adalimumab | 11 | 11-300 | ~6 hrs | Application |
| | | | | | based |
| ELISA | Trastuzumab | 10 | 30-1,000 | ~6 hrs | Application |
| | | | | | based |
| Lateral | Adalimumab | 800 | 1300- | ~15 min | 20-fold |
| Flow | | | 35,000 | (per | |
| | | | | sample) | |
| HMSA | Adalimumab | 18 | 120-1,600 | ~3 hrs | Extensive |
| (Homo- | | | ug/mL | (per | sample |
| genous | | | | sample) | preparation |
| Mobility | | | | | |
| Shift | | | | | |
| Assay) | | | | | |
| Peptide | Trastuzumab | 5000 | 10,000- | ~5 hours | 500-fold |
| ELISA | | | 180,000 | | |
| UPLC | Trastuzumab | 0.74 | 0.03- | ~4 hours | Extensive |
| MRM- | | | 42180 | (per | sample |
| MS | | | | sample) | preparation |

Raw data corresponding to the embodiments described in the above Examples are provided in the Tables below.

TABLE 7
Raw emission data of the dual fluorescence affinity complex
Trastuzumab:2PrL:2Her2 formed by mixing ^{Rh}PrL and ^{Fl}Her2
in PBS with an excitation at 480 nm and reading at 525 nm
for each concentration of antibody. Incubation time was 20
minutes. Concentrations were done in triplicates (at minimum)
to measure the error. This data was normalized and used to
perform statistical analysis on the results.

| Antibody | | | | | | |
|---|---|---|---|---|---|---|
| Concentration | | | | | | |
| (nM) | Emission Intensity | | | | | |
| 0 | 9359 | 9544 | 9547 | 9852 | 10130 | 9949 |
| 0.92 | 9277 | 9190 | 9264 | 9725 | 9644 | 9622 |
| 1.15 | 9191 | 9031 | 9183 | — | — | — |
| 1.47 | 8843 | 8953 | 9087 | — | — | — |
| 1.84 | 8957 | 8765 | 8925 | 9199 | 9123 | 9011 |
| 2.3 | 8723 | 8742 | 8669 | — | — | — |
| 2.75 | 8535 | 8584 | 8543 | — | — | — |
| 3.67 | 8155 | 8088 | 8150 | 8516 | 8550 | 8262 |
| 4.59 | 7897 | 7806 | 7664 | — | — | — |
| 5.51 | 7736 | 7673 | 7580 | — | — | — |
| 5.97 | 7627 | 7411 | 7340 | 8039 | 7961 | 7954 |
| 6.7 | 7310 | 7045 | 7050 | 7662 | 7518 | 7487 |
| 7.34 | 7263 | 7261 | 7162 | 7781 | 7599 | 7610 |
| 9.18 | 7130 | 6845 | 6896 | 7124 | 7036 | 7026 |
| 10 | 7414 | 7233 | 7280 | — | — | — |
| 10.6 | 7429 | 7240 | 7281 | — | — | — |
| 11.2 | 7175 | 7079 | 7026 | — | — | — |
| 11.6 | 6903 | 6836 | 6783 | — | — | — |
| 12.3 | 6497 | 6417 | 6365 | 6947 | 6818 | 6890 |
| 13.6 | 6839 | 6940 | 6860 | — | — | — |
| 15.9 | 6747 | 6716 | 6856 | — | — | — |
| 18.4 | 6303 | 6348 | 6241 | 6793 | 6778 | 6440 |

TABLE 8
Raw emission data of the dual fluorescence affinity complex
Trastuzumab:2PrL:2Her2 formed by mixing ^{Rh}PrL and ^{Fl}Her2
in CHO with an excitation at 480 nm and reading at 525 nm
for each concentration of antibody. Incubation time was 20
minutes. Concentrations were done in triplicates (at minimum)
to measure the error. This data was normalized and used to
perform statistical analysis on the results.

| Antibody | | | | | | |
|---|---|---|---|---|---|---|
| Concentration | | | | | | |
| (nM) | Emission Intensity | | | | | |
| 0 | 18755 | 18347 | 18129 | 12337 | 12335 | 12279 |
| 0.92 | 18046 | 18154 | 18237 | 11983 | 12066 | 12213 |
| 1.15 | 17736 | 17779 | 17495 | — | — | — |
| 1.47 | 17666 | 17648 | 17067 | — | — | — |
| 1.84 | 18206 | 18013 | 16989 | 11457 | 11609 | 11414 |
| 2.3 | 16108 | 16116 | 16243 | — | — | — |
| 2.75 | 16635 | 16370 | 16082 | — | — | — |
| 3.67 | 15950 | 15826 | 15359 | 11078 | 10855 | 10811 |
| 4.59 | 16715 | 15579 | 15576 | — | — | — |
| 5.51 | 15473 | 14993 | 15415 | — | — | — |
| 5.97 | 14726 | 14716 | 14924 | 9843 | 9853 | 9930 |
| 6.7 | 15498 | 14849 | 14677 | 9861 | 9691 | 9683 |
| 7.34 | 15464 | 14784 | 14760 | 9661 | 9604 | 9468 |
| 9.18 | 14398 | 14299 | 14188 | 9063 | 9069 | 8983 |
| 10 | 9003 | 8829 | 8857 | — | — | — |
| 10.6 | 9028 | 8792 | 8876 | — | — | — |
| 11.2 | 9028 | 8893 | 8944 | — | — | — |
| 11.6 | 8790 | 8802 | 8702 | — | — | — |
| 12.3 | 13806 | 13700 | 13684 | 8629 | 8497 | 8642 |
| 13.6 | 8627 | 8643 | 8455 | — | — | — |
| 15.9 | 8684 | 8584 | 8677 | — | — | — |
| 18.4 | 12877 | 12866 | 12680 | 8832 | 8759 | 8714 |

TABLE 9
Raw emission data of the dual fluorescence affinity complex
Adalimumab:2PrL:2TNF-α formed by mixing ^{Rh}PrL and ^{Fl}TNF-α
in PBS with an excitation at 480 nm and reading at 525 nm
for each concentration of antibody. Incubation time was 20
minutes. Concentrations were done in triplicates (at minimum)
to measure the error. This data was normalized and used to
perform statistical analysis on the results.

| Antibody | | | | | | |
|---|---|---|---|---|---|---|
| Concentration | | | | | | |
| (nM) | Emission Intensity | | | | | |
| 0 | 8847 | 8890 | 9029 | 8224 | 8140 | 8246 |
| 0.89 | 8391 | 8182 | 8350 | 7780 | 7606 | 7583 |
| 1.11 | 8231 | 8175 | 8328 | — | — | — |
| 1.43 | 8204 | 8087 | 8131 | — | — | — |
| 1.78 | 7849 | 7762 | 7748 | 7203 | 7127 | 7072 |
| 2.23 | 7477 | 7265 | 7322 | — | — | — |
| 2.67 | 7225 | 7014 | 7176 | — | — | — |
| 3.56 | 6576 | 6584 | 6565 | 6215 | 6064 | 6177 |
| 4.45 | 6158 | 6077 | 6054 | — | — | — |
| 5.34 | 5757 | 5590 | 5530 | — | — | — |
| 5.79 | 5167 | 5099 | 5076 | 4727 | 4506 | 4638 |
| 6.5 | 4856 | 4735 | 4917 | 4239 | 4107 | 4244 |
| 7.13 | 4526 | 4301 | 4198 | 3953 | 3791 | 3835 |
| 8.91 | 3442 | 3252 | 3246 | 3208 | 2994 | 2925 |
| 9.75 | 2592 | 2334 | 2480 | — | — | — |
| 10.3 | 2400 | 2164 | 2210 | — | — | — |
| 10.9 | 2296 | 2142 | 2126 | — | — | — |
| 11.3 | 2069 | 2018 | 1928 | — | — | — |
| 11.9 | 2508 | 2250 | 2160 | 1777 | 1697 | 1705 |
| 13.2 | 1691 | 1560 | 1634 | — | — | — |
| 15.4 | 1476 | 1522 | 1514 | — | — | — |
| 17.8 | 1570 | 1573 | 1504 | 1476 | 1511 | 1460 |

TABLE 10
Raw emission data of the dual fluorescence affinity complex
Adalimumab:2PrL:2TNF-α formed by mixing ^{Rh}PrL and ^{Fl}TNF-α
in CHO with an excitation at 480 nm and reading at 525 nm
for each concentration of antibody. Incubation time was 20
minutes. Concentrations were done in triplicates (at minimum)
to measure the error. This data was normalized and used to
perform statistical analysis on the results.

| Antibody | | | | | | |
|---|---|---|---|---|---|---|
| Concentration | | | | | | |
| (nM) | Emission Intensity | | | | | |
| 0 | 12405 | 12553 | 12432 | 12310 | 12521 | 12647 |
| 0.89 | 11582 | 11659 | 11606 | 11511 | 11452 | 11570 |
| 1.11 | 11522 | 11624 | 11233 | — | — | — |
| 1.43 | 11275 | 10909 | 10953 | — | — | — |
| 1.78 | 10921 | 10910 | 10851 | 10873 | 10903 | 10740 |
| 2.23 | 10378 | 10418 | 10412 | — | — | — |
| 2.67 | 10158 | 10045 | 10107 | — | — | — |
| 3.56 | 9580 | 9305 | 9364 | 9432 | 9291 | 9253 |
| 4.45 | 8989 | 9324 | 8983 | — | — | — |
| 5.34 | 8777 | 8490 | 8438 | — | — | — |
| 5.79 | 7869 | 8186 | 7882 | 7940 | 7892 | 8006 |
| 6.5 | 7319 | 7332 | 7112 | 7483 | 7426 | 7445 |
| 7.13 | 7210 | 7241 | 7304 | 7196 | 7233 | 7233 |
| 8.91 | 6368 | 5953 | 6096 | 5928 | 5912 | 5858 |
| 9.75 | 5855 | 5624 | 5691 | — | — | — |
| 10.3 | 5283 | 5260 | 5182 | — | — | — |
| 10.9 | 4995 | 4863 | 4863 | — | — | — |
| 11.3 | 4665 | 4648 | 4623 | — | — | — |
| 11.9 | 4924 | 4581 | 4631 | 4529 | 4566 | 4497 |
| 13.2 | 4378 | 4286 | 4274 | — | — | — |
| 15.4 | 4010 | 4026 | 3957 | — | — | — |
| 17.8 | 3117 | 3023 | 3034 | 3656 | 3534 | 3524 |

TABLE 11
Raw emission data of the dual fluorescence affinity complex
Adalimumab:2PrL:2TNF-α formed by mixing ^{Rh}PrL and ^{Fl}TNF-α
in PBS with an excitation at 480 nm and reading at 525 nm
for each concentration of antibody. Incubation time was 10
minutes. Concentrations were done in triplicates (at minimum)
to measure the error. This data was normalized and used to
perform statistical analysis on the results.

| Antibody | | | | | | |
|---|---|---|---|---|---|---|
| Concentration | | | | | | |
| (nM) | Emission Intensity | | | | | |
| 0 | 8943 | 8914 | 9057 | 8408 | 8320 | 8350 |
| 0.89 | 8449 | 8377 | 8467 | 7803 | 7716 | 7945 |
| 1.11 | 8262 | 8155 | 8399 | — | — | — |
| 1.43 | 8270 | 8035 | 8111 | — | — | — |
| 1.78 | 7909 | 7830 | 7812 | 7409 | 7253 | 7195 |
| 2.23 | 7393 | 7307 | 7393 | — | — | — |
| 2.67 | 7171 | 6970 | 6888 | — | — | — |
| 3.56 | 6440 | 6384 | 6517 | 6406 | 6163 | 6110 |
| 4.45 | 6065 | 6080 | 6021 | — | — | — |
| 5.34 | 5617 | 5614 | 5495 | — | — | — |
| 5.79 | 5204 | 4972 | 5047 | 4543 | 4260 | 4484 |
| 6.5 | 4928 | 4594 | 4732 | 4326 | 3873 | 4126 |
| 7.13 | 4505 | 4270 | 4243 | 3943 | 3821 | 3848 |
| 8.91 | 3404 | 3192 | 3312 | 3106 | 2925 | 3006 |
| 9.75 | 2730 | 2524 | 2739 | — | — | — |
| 10.3 | 2681 | 2466 | 2516 | — | — | — |
| 10.9 | 2609 | 2408 | 2331 | — | — | — |
| 11.3 | 2201 | 2201 | 2025 | — | — | — |
| 11.9 | 2498 | 2352 | 2273 | 1907 | 1866 | 1822 |
| 13.2 | 1917 | 1774 | 1705 | — | — | — |
| 15.4 | 1646 | 1605 | 1612 | — | — | — |
| 17.8 | 1646 | 1548 | 1595 | 1527 | 1537 | 1489 |

TABLE 12
Raw emission data of the dual fluorescence affinity complex
Adalimumab:2PrL:2TNF-α formed by mixing ^{Rh}PrL and ^{Fl}TNF-α
in PBS with an excitation at 480 nm and reading at 525 nm
for each concentration of antibody. Incubation time was 5
minutes. Concentrations were done in triplicates (at minimum)
to measure the error. This data was normalized and used to
perform statistical analysis on the results.

| Antibody | | | | | | |
|---|---|---|---|---|---|---|
| Concentration | | | | | | |
| (nM) | Emission Intensity | | | | | |
| 0 | 8905 | 9109 | 8893 | 8653 | 8806 | 8612 |
| 0.89 | 8381 | 8313 | 8305 | 8031 | 8019 | 8107 |
| 1.11 | 8285 | 8337 | 8386 | — | — | — |
| 1.43 | 8252 | 8310 | 8216 | — | — | — |
| 1.78 | 7824 | 7826 | 7746 | 7662 | 7540 | 7508 |
| 2.23 | 7444 | 7340 | 7350 | — | — | — |
| 2.67 | 7128 | 6953 | 7060 | — | — | — |
| 3.56 | 6532 | 6429 | 6453 | 6607 | 6653 | 6435 |
| 4.45 | 6042 | 5910 | 5864 | — | — | — |
| 5.34 | 5679 | 5381 | 5400 | — | — | — |
| 5.79 | 5177 | 5015 | 4977 | 4960 | 4863 | 4657 |
| 6.5 | 4767 | 4660 | 4760 | 4521 | 4571 | 4525 |
| 7.13 | 4437 | 4212 | 4211 | 4303 | 4051 | 4244 |
| 8.91 | 3486 | 3297 | 3310 | 3723 | 3200 | 3479 |
| 9.75 | 3152 | 2778 | 3107 | — | — | — |
| 10.3 | 2759 | 2786 | 2870 | — | — | — |
| 10.9 | 2973 | 2650 | 2682 | — | — | — |
| 11.3 | 2568 | 2508 | 2537 | — | — | — |
| 11.9 | 2600 | 2405 | 2294 | 2335 | 2132 | 2126 |
| 13.2 | 2194 | 1969 | 1985 | — | — | — |
| 15.4 | 1888 | 1720 | 1758 | — | — | — |
| 17.8 | 1704 | 1631 | 1676 | 1644 | 1642 | 1613 |

TABLE 13
Raw emission data of the dual fluorescence affinity complex
Adalimumab:2PrL:2TNF-α formed by mixing ^{Rh}PrL and ^{Fl}TNF-α
in PBS with an excitation at 480 nm and reading at 525 nm
for each concentration of antibody. Incubation time was 1
minute. Concentrations were done in triplicates (at minimum)
to measure the error. This data was normalized and used to
perform statistical analysis on the results.

| Antibody | | | | | | |
|---|---|---|---|---|---|---|
| Concentration | | | | | | |
| (nM) | Emission Intensity | | | | | |
| 0 | 9305 | 9481 | 9388 | 9165 | 9068 | 9103 |
| 0.89 | 8940 | 8801 | 8974 | 8771 | 8615 | 8527 |
| 1.11 | 8626 | 8649 | 8594 | — | — | — |
| 1.43 | 8643 | 8629 | 8526 | — | — | — |
| 1.78 | 8107 | 8207 | 8293 | 8133 | 8059 | 8132 |
| 2.23 | 7899 | 7922 | 7788 | — | — | — |
| 2.67 | 7742 | 7345 | 7500 | — | — | — |
| 3.56 | 7022 | 6847 | 6817 | 7053 | 6894 | 6938 |
| 4.45 | 6347 | 6254 | 6174 | — | — | — |
| 5.34 | 6031 | 5853 | 5823 | — | — | — |
| 5.79 | 5575 | 5254 | 5277 | 6122 | 5884 | 5774 |
| 6.5 | 5139 | 5012 | 5128 | 5627 | 5552 | 5511 |
| 7.13 | 4863 | 4611 | 4621 | 5335 | 5110 | 5293 |
| 8.91 | 3909 | 3829 | 3814 | 4854 | 4394 | 4564 |
| 9.75 | 4447 | 4105 | 4265 | — | — | — |
| 10.3 | 4155 | 4073 | 4048 | — | — | — |
| 10.9 | 4076 | 3950 | 3910 | — | — | — |
| 11.3 | 4015 | 3842 | 3797 | — | — | — |
| 11.9 | 3134 | 2867 | 2825 | 3991 | 3769 | 3677 |
| 13.2 | 3783 | 3492 | 3513 | — | — | — |
| 15.4 | 3241 | 2984 | 2957 | — | — | — |
| 17.8 | 1969 | 1983 | 1986 | 2642 | 2582 | 2545 |

TABLE 14
Raw emission data of the dual fluorescence affinity complex
Adalimumab:2PrL:2TNF-α formed by mixing ^{Rh}PrL and ^{Fl}TNFA-α
in CHO with an excitation at 480 nm and reading at 525 nm
for each concentration of antibody. Incubation time was 10
minutes. Concentrations were done in triplicates (at minimum)
to measure the error. This data was normalized and used to
perform statistical analysis on the results.

| Antibody | | | | | | |
|---|---|---|---|---|---|---|
| Concentration | | | | | | |
| (nM) | Emission Intensity | | | | | |
| 0 | 12620 | 12407 | 12646 | 12685 | 12688 | 12593 |
| 0.89 | 11739 | 11588 | 11572 | 11664 | 11625 | 11741 |
| 1.11 | 11681 | 11815 | 11399 | — | — | — |
| 1.43 | 11256 | 10965 | 10752 | — | — | — |
| 1.78 | 10940 | 10941 | 10894 | 10624 | 10885 | 10705 |
| 2.23 | 10343 | 10334 | 10405 | — | — | — |
| 2.67 | 10225 | 10056 | 9990 | — | — | — |
| 3.56 | 9565 | 9285 | 9462 | 9407 | 9309 | 9278 |
| 4.45 | 9121 | 9505 | 9119 | — | — | — |
| 5.34 | 8816 | 8433 | 8589 | — | — | — |
| 5.79 | 8017 | 8375 | 7930 | 7839 | 7869 | 7755 |
| 6.5 | 7492 | 7325 | 7350 | 7366 | 7317 | 7394 |
| 7.13 | 7263 | 7342 | 7251 | 7310 | 6979 | 6978 |
| 8.91 | 6502 | 5949 | 6296 | 5933 | 5904 | 5747 |
| 9.75 | 5790 | 5620 | 5700 | — | — | — |
| 10.3 | 5529 | 5524 | 5434 | — | — | — |
| 10.9 | 5133 | 5270 | 5048 | — | — | — |
| 11.3 | 5253 | 4832 | 4634 | — | — | — |
| 11.9 | 5081 | 4707 | 4912 | 4686 | 4694 | 4614 |
| 13.2 | 4560 | 4237 | 4446 | — | — | — |
| 15.4 | 4165 | 4146 | 4155 | — | — | — |
| 17.8 | 3325 | 3252 | 3084 | 3780 | 3684 | 3743 |

TABLE 15
Raw emission data of the dual fluorescence affinity complex
Adalimumab:2PrL:2TNF-α formed by mixing ^{Rh}PrL and ^{Fl}TNFA-α
in CHO with an excitation at 480 nm and reading at 525 nm
for each concentration of antibody. Incubation time was 5
minutes. Concentrations were done in triplicates (at minimum)
to measure the error. This data was normalized and used to
perform statistical analysis on the results.

| Antibody | | | | | | |
|---|---|---|---|---|---|---|
| Concentration | | | | | | |
| (nM) | Emission Intensity | | | | | |
| 0 | 12879 | 12912 | 12815 | 12964 | 12967 | 12938 |
| 0.89 | 11813 | 12157 | 12098 | 11945 | 12134 | 11924 |
| 1.11 | 11864 | 11885 | 11507 | — | — | — |
| 1.43 | 11672 | 11267 | 11044 | — | — | — |
| 1.78 | 11214 | 11392 | 11117 | 11191 | 11088 | 11080 |
| 2.23 | 10433 | 10566 | 10677 | — | — | — |
| 2.67 | 10204 | 10386 | 10362 | — | — | — |
| 3.56 | 9853 | 9600 | 9660 | 9796 | 9427 | 9403 |
| 4.45 | 9165 | 9454 | 9618 | — | — | — |
| 5.34 | 8978 | 8427 | 8655 | — | — | — |
| 5.79 | 8029 | 8514 | 8082 | 7943 | 7743 | 8019 |
| 6.5 | 7517 | 7612 | 7266 | 7424 | 7370 | 7489 |
| 7.13 | 7351 | 7523 | 7357 | 7276 | 7111 | 7307 |
| 8.91 | 6697 | 6220 | 6455 | 6216 | 6180 | 5991 |
| 9.75 | 6129 | 6235 | 6285 | — | — | — |
| 10.3 | 5962 | 6082 | 6077 | — | | — |
| 10.9 | 5528 | 5618 | 5629 | — | — | — |
| 11.3 | 5722 | 5361 | 4938 | — | — | — |
| 11.9 | 5209 | 4819 | 5129 | 4755 | 4896 | 5077 |
| 13.2 | 4832 | 4803 | 4690 | — | — | — |
| 15.4 | 4458 | 4556 | 4488 | — | — | — |
| 17.8 | 3584 | 3286 | 3310 | 4138 | 3999 | 3963 |

TABLE 16
Raw emission data of the dual fluorescence affinity complex
Adalimumab:2PrL:2TNF-α formed by mixing ^{Rh}PrL and ^{Fl}TNF-α
in CHO with an excitation at 480 nm and reading at 525 nm
for each concentration of antibody. Incubation time was 1
minute. Concentrations were done in triplicates (at minimum)
to measure the error. This data was normalized and used to
perform statistical analysis on the results.

| Antibody | | | | | | |
|---|---|---|---|---|---|---|
| Concentration | | | | | | |
| (nM) | Emission Intensity | | | | | |
| 0 | 12808 | 12903 | 12912 | 13224 | 13496 | 13350 |
| 0.89 | 12084 | 12290 | 12045 | 12602 | 12510 | 12890 |
| 1.11 | 12147 | 12014 | 11892 | — | — | — |
| 1.43 | 11628 | 11416 | 11444 | — | — | — |
| 1.78 | 11486 | 11532 | 11462 | 12024 | 11755 | 12005 |
| 2.23 | 10863 | 10896 | 10954 | — | — | — |
| 2.67 | 10715 | 10752 | 10589 | — | — | — |
| 3.56 | 10241 | 9981 | 9941 | 10714 | 10246 | 10499 |
| 4.45 | 9356 | 9729 | 9414 | — | — | — |
| 5.34 | 9417 | 8761 | 9089 | — | — | — |
| 5.79 | 8424 | 8821 | 8395 | 9535 | 9504 | 9610 |
| 6.5 | 7727 | 7926 | 7449 | 9401 | 9339 | 9381 |
| 7.13 | 7619 | 7685 | 7598 | 9215 | 8809 | 8949 |
| 8.91 | 7256 | 6818 | 6766 | 8036 | 7943 | 7857 |
| 9.75 | 7995 | 7837 | 7780 | — | — | — |
| 10.3 | 7543 | 7613 | 7692 | — | — | — |
| 10.9 | 7465 | 7404 | 7302 | — | — | — |
| 11.3 | 7491 | 7084 | 7126 | — | — | — |
| 11.9 | 5701 | 5360 | 5496 | 7143 | 7166 | 7063 |
| 13.2 | 7044 | 6727 | 7033 | — | — | — |
| 15.4 | 6666 | 6512 | 6346 | — | — | — |
| 17.8 | 3964 | 3772 | 3735 | 5884 | 5544 | 5723 |

TABLE 17
Raw emission data of the dual fluorescence affinity complex
Trastuzumab:2Her2 formed by mixing ^{Fl}Her2 in PBS with an
excitation at 480 nm and reading at 525 nm for each concentration
of antibody. Incubation time was 20 minutes. Concentrations
were done in triplicates to measure the error.
This data was normalized and used to perform
statistical analysis on the results.

| Antibody | | | |
|---|---|---|---|
| Concentration | | | |
| (nM) | Emission Intensity | | |
| 0 | 287 | 282 | 331 |
| 0.92 | 288 | 302 | 337 |
| 1.15 | 263 | 304 | 299 |
| 1.47 | 300 | 268 | 278 |
| 1.84 | 257 | 276 | 277 |
| 2.3 | 266 | 263 | 290 |
| 2.75 | 264 | 263 | 284 |
| 3.67 | 273 | 245 | 275 |
| 4.59 | 261 | 246 | 282 |
| 5.51 | 250 | 275 | 266 |
| 5.97 | 267 | 253 | 286 |
| 6.7 | 272 | 268 | 271 |
| 7.34 | 294 | 291 | 253 |
| 9.18 | 276 | 262 | 290 |
| 12.3 | 253 | 270 | 278 |
| 18.4 | 261 | 291 | 277 |

TABLE 18
Raw emission data of the dual fluorescence affinity complex
Trastuzumab:2PrL formed by mixing ^{Rh}PrL in PBS with an
excitation at 480 nm and reading at 573 nm for each concentration
of antibody. Incubation time was 20 minutes. Concentrations
were done in triplicates to measure the error. This data was
normalized and used to perform statistical analysis on the results.

| Antibody | | | |
|---|---|---|---|
| Concentration | | | |
| (nM) | Emission Intensity | | |
| 0 | 9807 | 9716 | 9904 |
| 0.92 | 9774 | 9865 | 9925 |
| 1.15 | 9935 | 10075 | 10049 |
| 1.47 | 9991 | 9837 | 9789 |
| 1.84 | 9281 | 9289 | 9190 |
| 2.3 | 9171 | 9120 | 9204 |
| 2.75 | 9346 | 9156 | 9299 |
| 3.67 | 9199 | 9207 | 9196 |
| 4.59 | 8846 | 8984 | 8936 |
| 5.51 | 8909 | 8905 | 8784 |
| 5.97 | 8734 | 8817 | 8893 |
| 6.7 | 8980 | 9031 | 8984 |
| 7.34 | 8908 | 8850 | 8980 |
| 9.18 | 8922 | 8842 | 8788 |
| 12.3 | 8966 | 8880 | 8819 |
| 18.4 | 8704 | 8733 | 8709 |

TABLE 19
Raw emission data of the fluorescence affinity complex
Adalimumab:2TNF-α formed by mixing ^{Fl}TNF-α in PBS
with an excitation at 480 nm and reading at 525 nm for each
concentration of antibody. Incubation time was 20 minutes.
Concentrations were done in triplicates to
measure the error. This data was normalized
and used to perform statistical analysis on the results.

| Antibody | | | |
|---|---|---|---|
| Concentration | | | |
| (nM) | Emission Intensity | | |
| 0 | 9540 | 9832 | 9871 |
| 0.891 | 9647 | 9663 | 9747 |
| 1.11 | 9802 | 9771 | 9795 |
| 1.43 | 9801 | 9450 | 9314 |
| 1.78 | 9653 | 9919 | 9904 |
| 2.23 | 9817 | 10000 | 9980 |
| 2.67 | 9893 | 9760 | 9663 |
| 3.56 | 9700 | 9611 | 9305 |
| 4.45 | 9319 | 9529 | 9682 |
| 5.34 | 9481 | 9559 | 9507 |
| 5.79 | 10640 | 9373 | 9440 |
| 6.5 | 9437 | 9211 | 9076 |
| 7.13 | 9058 | 9226 | 13273 |
| 8.91 | 9140 | 9141 | 8959 |
| 11.9 | 8806 | 8806 | 8670 |
| 17.8 | 8861 | 8630 | 8558 |

TABLE 20
Raw emission data of the dual fluorescence affinity complex
Adalimumab:2PrL formed by mixing ^{Rh}PrL in PBS with an
excitation at 480 nm and reading at 573 nm for each concentration of
antibody. Incubation time was 20 minutes. Concentrations
were done in triplicates to measure the error. This data was
normalized and used to perform statistical analysis on the results.

| Antibody | | | |
|---|---|---|---|
| Concentration | | | |
| (nM) | Emission Intensity | | |
| 0 | 634 | 550 | 537 |
| 0.891 | 565 | 499 | 567 |
| 1.11 | 518 | 523 | 508 |
| 1.43 | 557 | 540 | 507 |
| 1.78 | 523 | 576 | 545 |
| 2.23 | 551 | 546 | 556 |
| 2.67 | 563 | 578 | 537 |
| 3.56 | 549 | 533 | 531 |
| 4.45 | 558 | 543 | 557 |
| 5.34 | 554 | 596 | 561 |
| 5.79 | 562 | 552 | 546 |
| 6.5 | 585 | 578 | 554 |
| 7.13 | 489 | 535 | 557 |
| 8.91 | 548 | 528 | 574 |
| 11.9 | 539 | 553 | 562 |
| 17.8 | 535 | 569 | 543 |

What is claimed is:

1. A ratiometric bioassay method for detecting a target antibody in a sample, the method comprising:
combining an antigen coupled to a first fluorophore, an antibody-binding moiety coupled to a second fluorophore, and a sample comprising or suspected of comprising a target antibody;
exposing the antigen coupled to the first fluorophore, the antibody-binding moiety coupled to the second fluorophore, and the sample to fluorescent light comprising an excitation wavelength of the first and/or the second fluorophore; and
detecting fluorescence emission from the first and/or the second fluorophore;
wherein the fluorescence emission from the first and the second fluorophore is reduced, and wherein the reduced fluorescence emission is proportional to the antibody concentration in the sample.

2. The bioassay method according to claim 1, wherein the antigen is capable of being bound by the antibody in the sample.

3. The bioassay method according to claim 1 or 2, wherein the antibody-binding moiety comprises a polypeptide capable of binding a region of the target antibody that does not comprise the antigen-binding site.

4. The bioassay method according to any of claims 1 to 3, wherein the antibody binding moiety comprises Protein L, Protein A, or Protein G.

5. The bioassay method according to any of claims 1 to 3, wherein the antibody binding moiety is Protein L.

6. The bioassay method according to any of claims 1 to 5, wherein the first fluorophore is a high-energy fluorophore and the second fluorophore is a low-energy fluorophore.

7. The bioassay method according to any of claims 1 to 5, wherein the second fluorophore is a high-energy fluorophore and the first fluorophore is a low-energy fluorophore.

8. The bioassay method according to any of claims 1 to 7, wherein the high-energy fluorophore is selected from the group consisting of Fluorescein, Oregon Green 488, Oregon Green 514, Rhodamine Green, Rhodamine Green-X, Eosin, 4′, 6-diamidino-2-phenylindole, Alexafluor 405, Alexafluor 350, Alexafluor 500, Alexafluor 488, Alexafluor 430, Alexafluor 514, and Alexafluor 532.

9. The bioassay method according to any of claims 1 to 8, wherein the low-energy fluorophore is selected from the group consisting of Rhodamine, Rhodamine B, Rhodamine Red-X, Tetramethylrhodamine, Lissamine, Texas Red and Texas Red-X, Naphthofluorescein, Carboxyrhodamine 6G, Alexafluor 555, Alexafluor 546, Alexafluor 568, Alexafluor 594, Alexafluor 610, Alexafluor 633, Alexafluor 635, Alexafluor 647, Alexafluor 660, Alexafluor 680, Alexafluor 700, and Alexafluor 750.

10. The bioassay method according to any of claims 1 to 9, wherein the antigen is coupled to fluorescein or Alexafluor 350.

11. The bioassay method according to any of claims 1 to 10, wherein the antibody-binding moiety is coupled to Rhodamine.

12. The bioassay method according to any of claims 1 to 11, wherein the method further comprises determining the concentration of the target antibody in the sample based on the reduction in fluorescence emission of the first and/or the second fluorophore.

13. The bioassay method according to any of claims 1 to 12, wherein the method further comprises incubating the sample comprising or suspected of comprising the target antibody, the antigen coupled to the first fluorophore, and the antibody-binding moiety coupled to the second fluorophore for 30 minutes or less prior to measuring the fluorescent emission of the first and/or the second fluorophore.

14. The bioassay method according to any of claims 1 to 12, wherein the method further comprises incubating the sample comprising or suspected of comprising the target antibody, the antigen coupled to the first fluorophore, and the antibody-binding moiety coupled to the second fluorophore for 10 minutes or less prior to measuring the fluorescent emission of the first and/or the second fluorophore.

15. The bioassay method according to any of claims 1 to 14, wherein the antigen is coupled to the first fluorophore at an antigen-to-fluorophore ratio of about 1:0.2 to about 1:20.

16. The bioassay method according to any of claims 1 to 15, wherein the antibody-binding moiety is coupled to the second fluorophore at an moiety-to-fluorophore ratio of about 1:0.2 to about 1:20.

17. The bioassay method according to any of claims 1 to 16, wherein the first fluorophore and the second fluorophore are present in the composition at a first-to-second fluorophore ratio of about 1:1 to about 1:20.

18. The bioassay method according to any of claims 1 to 17, wherein the sample is undiluted.

19. The bioassay method according to any of claims 1 to 18, wherein the sample is a whole blood sample, a plasma sample, a serum sample, a urine sample, a saliva sample, a tissue sample, a cell culture sample, a cell lysate sample, or a cell culture media sample.

20. The bioassay method according to any of claims 1 to 19, wherein the antigen comprises the spike (S) protein of SARS-CoV-2, or a fragment thereof.

21. The bioassay method according to any of claims 1 to 19, wherein the antigen comprises the capsid protein of a viral vector for gene therapy, or a fragment thereof.

22. A composition for performing a ratiometric bioassay to detect an antibody in a sample, the composition comprising:
an antigen coupled to a first fluorophore;
an antibody-binding moiety coupled to a second fluorophore; and
a sample comprising or suspected of comprising a target antibody;
wherein fluorescence emission from the first and the second fluorophore is reduced upon exposure to fluorescent light comprising an excitation wavelength of the first and/or the second fluorophore, and wherein the reduced fluorescence emission is proportional to the antibody concentration in the sample.

23. The composition according to claim 22, wherein the antigen is capable of being bound by the antibody in the sample.

24. The composition according to claim 22 or claim 23, wherein the antibody-binding moiety comprises a polypeptide capable of binding a region of the target antibody that does not comprise the antigen-binding site.

25. The composition according to any of claims 22 to 24, wherein the antibody binding moiety comprises Protein L, Protein A, or Protein G.

26. The composition according to any of claims 22 to 25, wherein the first fluorophore is a high-energy fluorophore and the second fluorophore is a low-energy fluorophore.

27. The composition according to any of claims 22 to 26, wherein the second fluorophore is a high-energy fluorophore and the first fluorophore is a low-energy fluorophore.

28. The bioassay method according to any of claims 22 to 27, wherein the high-energy fluorophore is selected from the group consisting of Fluorescein, Oregon Green 488, Oregon Green 514, Rhodamine Green, Rhodamine Green-X, Eosin, 4′, 6-diamidino-2-phenylindole, Alexafluor 405, Alexafluor 350, Alexafluor 500, Alexafluor 488, Alexafluor 430, Alexafluor 514, and Alexafluor 532.

29. The composition according to any of claims 22 to 28, wherein the low-energy fluorophore is selected from the group consisting of Rhodamine, Rhodamine B, Rhodamine Red-X, Tetramethylrhodamine, Lissamine, Texas Red and Texas Red-X, Naphthofluorescein, Carboxyrhodamine 6G, Alexafluor 555, Alexafluor 546, Alexafluor 568, Alexafluor 594, Alexafluor 610, Alexafluor 633, Alexafluor 635, Alexafluor 647, Alexafluor 660, Alexafluor 680, Alexafluor 700, and Alexafluor 750.

30. The composition according to any of claims 22 to 29, wherein the antigen is coupled to the first fluorophore at an antigen-to-fluorophore ratio of about 1:0.5 to about 1:20.

31. The composition according to any of claims 22 to 30, wherein the antibody binding moiety is coupled to the second fluorophore at an moiety-to-fluorophore ratio of about 1:0.2 to about 1:20.

32. The composition according to any of claims 22 to 31, wherein the first fluorophore and the second fluorophore are present in the composition at a first-to-second fluorophore ratio of about 1:1 to about 1:20.

33. The composition according to any of claims 22 to 32, wherein the sample is undiluted.

34. The composition according to any of claims 22 to 33, wherein sample is a whole blood sample, a plasma sample, a serum sample, a urine sample, a saliva sample, a tissue sample, a cell culture sample, a cell lysate sample, or a cell culture media sample.

35. The composition according to any of claims 22 to 34, wherein the antigen comprises the spike (S) protein of SARS-CoV-2, or a fragment thereof.

36. The composition according to any of claims 22 to 34, wherein the antigen comprises the capsid protein of viral vector for gene therapy, or a fragment thereof.

37. A kit for performing a ratiometric bioassay to detect a target antibody in a sample, the kit comprising:
an antigen coupled to a first fluorophore;
an antibody binding moiety coupled to a second fluorophore; and
at least one container.

38. The kit according to claim 37, wherein the kit further comprises a buffer and/or instructions for performing the bioassay.

39. The kit according to claim 37 or claim 38, wherein the kit further comprises the target antibody or a fragment or derivative thereof.
